(19) United States
(12) Patent Application Publication
(10) Pub. No.: US 2015/0323536 A1
(43) Pub. Date: Nov. 12, 2015

(54) SATB1: A DETERMINANT OF MORPHOGENESIS AND TUMOR METASTATIS

(71) Applicant: The Regents of the University of California, Oakland, CA (US)

(72) Inventors: Terumi Kohwi-Shigematsu, El Cerrito, CA (US); Hye-Jung Han, Albany, CA (US); Yoshinori Kohwi, El Cerrito, CA (US)

(21) Appl. No.: 14/540,991

(22) Filed: Nov. 13, 2014

Related U.S. Application Data
(63) Continuation of application No. 12/058,574, filed on Mar. 28, 2008, now abandoned.
Application No. 12/058,574 is a continuation-in-part of application No. PCT/US2006/038711, filed on Oct. 2, 2006.
(60) Provisional application No. 60/722,833, filed on Sep. 30, 2005.

(51) Int. Cl. G01N 33/574; C12N 15/113; A61K 31/7105; C07K 16/18; C12N 15/115; C12Q 1/68; A61K 31/713

(52) U.S. Cl. CPC G01N 33/57415; C12Q 1/6886; C12N 15/113; A61K 31/713; C07K 16/18; C12N 15/115; A61K 31/7105; C12Q 2600/158; C12Q 2600/118; C12N 2310/14; C12N 2320/30; C12N 2310/11; C12N 2310/16; C07K 2317/76

(57) ABSTRACT

It is proposed that cancer cells express SATB1, and that SATB1 acts as a determinant for the acquisition of metastatic activity by controlling expression of a specific set of genes that promote metastatic activity. In order for cancer cells to gain the ability to metastasize, SATB1 re-organizes or re-packages genomic sequences in a specific manner to allow a switch in the pattern of gene expression. SATB1 expression was found restricted mainly to aggressive cancer cells where it may regulate the genetic and epigenetic changes that program the steps involved in the metastatic process. The present invention describes reagents and tools to detect the SATB1 protein for use in diagnosis and prognosis of aggressive cancers and therapeutics to inhibit SATB1 protein to deplete its expression in metastatic and aggressive cancers.

### CROSS-REFERENCE TO RELATED APPLICATIONS

This application in a continuation of application Ser. No. 12/058,574 filed Mar. 28, 2008, which is a continuation-in part of PCT Application No. PCT/US2006/038711 filed Oct. 2, 2006, which claims benefit of priority to U.S. Provisional Patent Application No. 60/722,833 filed on Sep. 30, 2005, each of which applications is hereby incorporated by reference in entirety for all purposes.

### STATEMENT OF GOVERNMENTAL SUPPORT

This invention was made during work supported by the U.S. Department of Energy at Lawrence Berkeley National Laboratory under Contract No. DE-AC02-05CH11231 and Grant No. R37 CA039681 awarded by the National Institutes of Health/National Cancer Institute. The government has certain rights in this invention.

### REFERENCE TO ATTACHED SEQUENCE LISTING AND TABLE APPENDIX

This application includes a Sequence Listing as a text file named “77429-006220US-924667 SEQ” created Apr. 24, 2015 and containing 4,988 bytes. The material contained in this text file is incorporated by reference in its entirety for all purposes.

This application incorporates by reference in its entirety, the attached table appendix found in paper form.

### BACKGROUND OF THE INVENTION

1. Field of the Invention

The present invention relates to cancer markers and therapeutics. More specifically, the present invention relates to the detection and inhibition of a general cancer marker which serves as an indicator of advanced stages of primary tumors and promotes aggressive cancers.

2. Related Art

Metastatic cells are a specialized subset of tumor cells within a primary tumor mass that have acquired the ability to disseminate from the site of the primary tumor and establish secondary tumors in distant organs. Different patterns of expression for a large number of genes have been correlated with breast cancer development and/or progression. Although different mutation events can ultimately lead to the development of metastatic breast cancer in different patients, there must be a common and fundamental molecular mechanism allowing breast carcinoma cells to acquire such an aggressive phenotype and to maintain it. It is likely that such a mechanism exists at the level of DNA organization in cells.

A cell must organize the enormous length of DNA into a tiny space of the cell's nucleus, in order to express only those genes relevant for that cell's function. Our recent work on the protein SATB1 in lymphocytes has shed light into the mystery of how this ‘functional’ packaging is accomplished. SATB1 organizes genomic DNA sequences by providing an intra-nuclear architecture, onto which a group of specialized DNA sequences are anchored and assembled, with those various enzymes and protein factors necessary for gene expression. Thus SATB1 acts as a genome organizer and controls numerous genes.

One of the inventors has been studying SATB1 for many years. SATB1 is described in U.S. Pat. No. 5,652,340 and antibodies made thereto are described in U.S. Pat. No. 5,869,621, which are hereby incorporated by reference. Our research group is studying how the genome is functionally organized in the nucleus. The recent work from Kohwi-Shigematsu's group on SATB1, which is expressed predominantly in the T cell lineage (Dickinson, L. A., T. Joh, Y. Kohwi, and T. Kohwi-Shigematsu, Cell 70:631, 1992; Alvarez, J. D., Yasui, D. H., Niida, H., Joh, T., Loh, D. Y., and Kohwi-Shigematsu, T., Genes Dev 14, 521-535, 2000), has introduced the novel concept that a single protein can provide a unique nuclear architecture onto which chromatin is folded by anchoring specialized DNA sequences (Yasui et al., Nature 419:641-645, 2002; Cai et al., Nat. Genet. 37:31-40, 2003). These specialized DNA sequences are called base unpairing regions (BURs), which are double-stranded DNA highly potentiated for base unpairing under negative superhelical strain (Kohwi-Shigematsu, T. and Kohwi, Y. Biochemistry, 29:9551-9560, 1990: Bode, J., Kohwi, Y., Dickinson, L. Joh, T Klehr, D., Mielke, C., and Kohwi-Shigematsu, T. Science, 255:195-197, 1992). SATB1 represents a new class of gene regulator: by targeting chromatin remodeling/modifying complexes to the DNA sequences anchored to the SATB1 nuclear architecture, it thus regulates chromatin structure over long distances as well as expression of numerous genes (Yasui, D., Miyano, M., Cai, S., Varga-Weisz, P., and Kohwi-Shigematsu, T. (2002) Nature 419, 641-645; Cai, S., Han, H. J., and Kohwi-Shigematsu, T. (2003) Nat Genet 34, 42-51).

It has never been expected that SATB1, which has been thought to be cell type-specific and necessary for T cell development, would also be expressed in breast cancer cells, primarily in metastatic breast cancer cells. We study SATB1, which is a cell-type specific nuclear protein which orchestrates the temporal and spatial expression of numerous genes during T cell differentiation (Alvarez, J. D., Yasui, D. H., Niida, H., Joh, T., Loh, D. Y., and Kohwi-Shigematsu, T. (2000) Genes Dev 14, 521-535). We have shown that SATB1 provides a unique cage-like nuclear architecture formed by SATB1 in thymocyte nuclei was also found in metastatic breast cancer nuclei. It was shown that SATB1 directly regulates genes which play key roles in T cell differentiation and function. Because SATB1 acts as a cell-specific genome organizer in T cells, it is highly likely that SATB1 also acts as a genome organizer in metastatic breast cancer and regulates key players necessary for the metastatic activity of breast cancer.

BURs that are targeted by SATB1 are also preferentially recognized by HMG-I(Y), SAF-A, PARP, and Ku70/86. These BUR-binding proteins also have elevated expression as cancer takes on a more aggressive phenotype. (Liu W-M, et al., HMG-I(Y) recognizes Base-unpairing regions of matrix attachment sequences and its increased expression is directly linked to metastatic breast cancer phenotype. Cancer Research 59, 5695-5703 (1999); Yanagisawa J., et al., A matrix attachment region (MAR)-binding activity due to a p114 kilodalton protein is found only in human breast carcinomas and not in normal and benign breast disease tissues. Cancer Research, 56, 457-462 (1996); Galande S and Kohwi-Shigematsu, T. Linking chromatin architecture to cellular phenotype: BUR-binding proteins in cancer. J. Cellular Biochem. Suppl. 35, 36-45 (2000)).

Metastasis is a multi-step process during which cancer cells disseminate from the site of primary tumors and establish secondary tumors in distant organs (Welch, D. R., Steeg, P. S., and Rinker-Schaeffer, C. W. (2000) Breast Cancer Res 2, 408-416). Recently, microarray analyses of various human tumor samples generated gene expression profiles that are potentially useful as prognostic markers of metastatic diseases (van de Vijver, M. J., et al., (2002) N Engl J Med 347, 1999-2009; Ramaswamy, S., and Perou, C. M. (2003) Lancet 361, 1576-1577; Sorlie, T., et al., (2003) Proc Natl Acad Sci USA 100, 8418-8423). The research in elucidating the specific contributions of such genes to tumor metastasis has been very difficult. Certain genes, however, have been found to promote metastatic phenotypes when ectopically expressed (Yang, J., et al., (2004) Cell 117, 927-939; Eckel, K. L., et al., (2003) DNA Cell Biol 22, 79-94). Tumor metastasis is the most common cause of death in cancer patients. Therefore, it is extremely important to identify key regulators of metastasis and their function, in order to devise effective interventions against metastasis in the future.

### SUMMARY OF THE INVENTION

Although the Special AT-rich binding protein 1 (SATB1) was originally characterized as a factor in the T cell lineage, SATB1 was unexpectedly found to be expressed in metastatic but not in non-metastatic breast cancer cell lines, and in human tissue specimens from advanced stages of breast carcinomas with metastasis. High levels of SATB1 expression was detected in all lymph-node positive, poorly differentiated infiltrating ductal carcinomas, and low-level expression in some, but not all, moderately differentiated tumor samples. SATB1 protein was detected in 23 of the 28 tumor samples examined, but it was undetected in all 10 normal controls. Of the 28 tumor samples, sixteen were metastatic breast carcinomas. SATB1 was expressed in all 16 metastatic breast carcinoma samples with very high statistical significance (P<0.0001) compared to either moderately differentiated tumor or normal tissue samples. SATB1 was not detected in any normal adjacent tissues. Furthermore, SATB1 was also found to be expressed in small lung cell carcinoma, leukemia (in Jurkat cells, CEM cells), lymphomas and colon cancers. Therefore, SATB1 may be a reliable marker for diagnosis and prognosis of cancer.

Furthermore, it will be possible to devise therapeutic strategies by targeting SATB1. Our new findings show that depletion of SATB1 from aggressive cancer cells reverses their aggressive phenotype to non-aggressive phenotype. Also, forced expression of SATB1 induces aggressive phenotypes in non-aggressive cancer cells. When SATB1 is expressed, this protein appears to promote metastatic activity of cancer cells by controlling expression of a specific set of genes which either promote or are necessary for such activity. It is thought that SATB1 contributes in reorganization of genomic sequences in a specific manner to allow a switch in the pattern of gene expression. The findings described herein show SATB1 acts as a determinant for cancer cells to switch to gain a new phenotype: viz. metastasis.

Thus, the methods and compositions described herein are both novel and useful in cancer research both with regards to developing a diagnostic/prognostic method and also therapeutic strategies. Tumor metastasis is the most common cause of death in cancer patients. Therefore, preventing cells from acquiring metastatic activity or promoting cell death for metastatic cells will save the lives of many cancer patients. Early detection of cells with a high index for metastasis at the initial stages of diagnosis will aid in identifying patients who will merit from an aggressive treatment regardless of their lymph node status. On the other hand, the absence of such cells in their tissue specimens will help to alleviate anxieties regarding recurrence.

### BRIEF DESCRIPTION OF THE DRAWINGS

FIG. 1A-1E shows photographs immunoblot analysis of SATB1 levels (a, upper panel) in normal mammary epithelial cells (HMEC), immortalized mammary epithelial cells (MCF-10A), non-aggressive breast cancer cell lines (BT474, MCF7, MDA-MB-435, SUM225 and SKBR3), and aggressive breast cancer cell lines (HCC202, Hs578T, MDA-MB-435, BT549, MDA-MB-231); α-tubulin loading control (FIG. 1A, middle panel). Transcript levels of SATB1, relative to GAPDH, determined by qRT-PCR and compared to Hs578T cells (FIG. 1A, bottom panel); error bars=s.e.m, n=3 experiments FIG. 1B Immunoblot analysis of SATB1 in representative human primary breast tumor specimen (top panel); α-tubulin loading control (bottom panel)). FIG. 1C Immunofluorescence images of poorly differentiated ductal carcinomas (top row) and adjacent normal tissues (bottom row) stained with anti-SATB1 and anti-cytokeratin 8 (CK8) or anti-cytoketatin 14 (CK14) antibodies, counterstained with DAPI (DNA; blue). Scale bars=30 μm. FIG. 1D SATB1 levels in representative tumor tissues (top, scale bar; 20 μm) and Kaplan-Meier plot (below) of overall survival of 985 patients with ductal breast carcinomas stratified by SATB1 expression level. Tissues scored as 0 (negative SATB1 nuclear staining for all tumor cells), 1 (positive SATB1 nuclear staining other than score 2) or 2 (moderate SATB1 staining for >50% tumor cells or strong staining for >5% tumor cells). Log-rank test showed significant differences between groups (P<0.001) FIG. 1E Relative multivariate significance of potential prognostic variables. Cox proportional hazards regression was used to test the independent prognostic contribution of SATB1 after accounting for other potentially important covariates.

FIG. 2 shows photographs of a Western blot analysis and quantitative RT-PCR analysis show that SATB1 expression was greatly reduced in MDA-MB-231 cells stably transfected with the pSUPER-puro construct expressing the shRNA against either the coding region (SATB1-shRNA1) or the 3′UTR (SATB1-shRNA2) compared to that in parental MDA-MB-231 cells. SATB1 expression levels remained unchanged in MDA-MB-231 cells that stably expressed an shRNA whose sequence did not match any known human gene (control shRNA).

FIG. 3 is pair of graphs showing the rate of proliferation of the parental MDA-MB-231 cells, control cells (expressing control shRNA), SATB1-shRNA1 MDA cells and SATB1-shRNA2 MDA cells grown on either plastic (2D) or matrigel (3D) culture plates was determined.

FIG. 4A shows unsupervised clustering (GeneSpring software) of genes differentially expressed between control-shRNA and SATB1-shRNA1 cells from both plastic and Matrigel culture conditions; 409 SATB1-activated genes and 456 SATB1-repressed genes are marked by double-headed arrows. Representative SATB1-activated (red) and SATB1-repressed genes (green) are either listed vertically or under each molecular pathway. Impact factor strength of SATB1-activated (red bars) and -repressed (green bars) genes is shown. Impact factor depictions have green as the top bars, red as the lower bars; red, control/SATB1-shRNA greater than 1.5/1.0; green, control/SATB1-shRNA less than 1.0/1.5 FIG. 4B is a chart showing the functional profiles of the genes regulated by SATB1 in MDA-MB-231 cells. Functional profiles using Gene Ontology terms for biological process and molecular function were constructed for SATB1-dependent up- and down-regulated genes (>2 fold) in either 2D or 3D cultures by Onto-Express using the initial pool of 20,000 genes (Codelink from Amersham) as the reference set.

FIG. 5 is photographs showing MDA-MB-231 cells were grown as a control (a) and MDA-MB-231 cells expressing SATB1 RNAi (b) were grown on plastic (2D) and on 3D culture (Matrigel) for 5 and 10 days.

FIG. 6 is photographs showing immunostaining with antibodies against F-actin, β-catenin, integrin α6 (all green), and counterstained with DAPI (DNA, blue) of SATB1-shRNA1 or SATB1-shRNA2 MDA cells grown in 3D culture which have an organized and polarized morphology, forming acinus-like structures.

FIG. 7A-7C are photographs showing that ectopic expression of SATB1 induces abnormal cell morphology. FIG. 7A MCF10A cells (vector control) and MCF10A-SB10 cells which ectopically express SATB1 were grown on plastic (2D) and on 3D Matrigel for 5 and 10 days. MCF10A-SB10 cells which ectopically express SATB1 exhibited an abnormal morphology as compared to the control cells which are an immortalized, non-tumorigenic cell line. FIG. 7B MCF-10A cells having an empty vector control, MCF10A-SB10 cells which ectopically express SATB1, were grown in 3D culture and then stained for nuclei (DAPI, blue) and α6 integrin (green). The MCF10A control cells with vector control exhibited normal morphology, while the MCF10A-SB10 cells expressing SATB1 exhibited an abnormal morphology (see four independent examples (i)-(iv)) as compared to normal MCF-10A cells (two independent examples (i) and (ii)). FIG. 7C MCF-10A cells having an empty vector control and MCF10A-SB10 cells which ectopically express SATB1, were grown in 3D culture and then stained for nuclei (DAPI, blue) and F-actin (red). The MCF10A control cells with vector control exhibited normal morphology, while the MCF10A-SB10 cells expressing SATB1 exhibited an abnormal morphology (see four independent examples (i)-(iv)) as compared to normal MCF-10A cells. (two independent examples (i) and (ii)).

FIG. 8A A soft agar assay was performed for MDA-MB-231 cells and MDA-MB-231 cells expressing SATB1 shRNA (top left panel) and for MCF10A cells (vector control) and MCF10A-SB10 cells which ectopically express SATB1 (bottom left panel). FIG. 8B The number of invasive cells were counted in the Boyden chamber invasion assay for each set of cells and graphed. MDA-MB-231 cells expressing SATB1 shRNA or shRNA2 showed decreased invasiveness to 20% compared with their host cells MDA-MB-231 cells (top right panel). In MCF10A cells and MCF10A cells with vector control there were 1-2 invasive cells, while with MCF10A-SB10 cells which ectopically express SATB1 there were 10-20 invasive cells (bottom right panel). Note: Very low number out of 4 millions shows invasive activity in MCF10A/SATB1. Nevertheless, these invasive cells were only found after forced expression of SATB1.

FIG. 9A-9C are photographs showing the effect of SATB1 on gene expression of other cell growth factors and cancer markers. FIG. 9A Semi-quantitative RT-PCR analyses were performed to assess the expression levels of the genes known to be involved in metastasis suppression or promotion. RT-PCR analyses were also performed for two independent Hs578T cell clones expressing control vector (control 1 and 2) and two independent cell clones (SATB1-1 and SATB-2) both carrying the SATB1 expression construct (pLXSN-SATB1) grown on 2D culture. PCR conditions were optimized to detect the SATB1-dependent up- or down-regulation of expression of each gene analyzed in either MDA-MB-231 cells or Hs578T cells. Host: no transfection, V: vector control, siRNA-1, siRNA-2; independent SATB1-depleted clones, SB10, SB12; independent SATB1 forced expressed clones and gene expression of 48 growth-related factors and cancer markers. FIG. 9B Protein expression levels of ERRB2 and β-catenin were analyzed by Western blot in parental MDA-MB-231 cells, control MDA-MB-231 cells (control shRNA), SATB1-shRNA1, and SATB1-shRNA2 MDA cells. GAPDH levels were used as loading control. FIG. 9C Semi-quantitative RT-PCR analyses showed that the expression level of genes shown here remained constant in all cell types. Expression levels of all other genes examined are shown

FIG. 10A-10B show that SATB1 targets ERBB2 gene locus in vivo to regulate ERBB2 expression in breast cancer cells. The SATB1 binding activity of each fragment was confirmed by an electrophoresis mobility shift assay (EMSA), using bacterially produced recombinant SATB1 protein These positions that show positive for EMSA, representing potential SATB1 binding sequence, are indicated by bars under the numbers. The fragments that actually bind to SATB1 in vivo are indicated by red star and also by red bar under ChIP. FIG. 10A A group of genes whose expression was changed by both shRNA-mediated removal of SATB1 in MDA-MB-231 cells and by SATB1-overexpression in Hs578T cells, compared to control cells FIG. 10B A group of genes whose expression was independent of SATB1 expression levels in cells. Gene structure is based on the data from USCS (URL http followed by: //genome.ucsc.edu/).

FIG. 11A shows photographs of lungs of nude mice which were injected with 1×10⁶ cells MDA-MB-231 cells expressing control-shRNA, SATB1-shRNA1 or SATB1-shRNA2 MDA cells. RNAi-mediated depletion of SATB1 inhibited the ability of MDA-MB-231 cells to metastasize to the lungs as compared to the control. The metastatic nodules are indicated by the arrows. FIG. 11B is a graph showing the total numbers of metastatic lung nodules from individual mice counted under a dissection microscope and the average number of metastatic nodules counted in each dissected lung. For lungs of representative mice indicated, human SATB1 expression levels in human breast cancer cells colonized in lungs were analyzed by RT-PCR using human GAPDH as a loading control, with the use of human SATB1 and GAPDH specific oligomers. The specificity of these oligomers for human genes is shown by the absence of RT-PCR signals for mouse thymcoytes (Thy) in the gel photograph shown. FIG. 11C shows photographs of lungs of nude mice which were injected with 2×10⁶ Hs578T cells transfected with vector alone (control) or with an SATB1 expression construct (pLXSN-SATB1) causing an overexpression of SATB1 which then promotes the ability of Hs578T cells to metastasize to lungs. Representative photos from three independent mice are shown. FIG. 11D is a graph showing the total number of metastatic lung nodules formed in lungs of mice injected with Hs578T cells that overexpress SATB1 (HS25) and controls (HS). Similar to FIG. 11B, human SATB1 expression was analyzed by RT-PCR for representative mice indicated.

FIG. 12 is photographs showing the forced expression of SATB1 in non-tumorigenic MCF-10A cells generated breast tumors in nude mice.

FIG. 13 is a cartoon showing the rationale resulting from the experiments—very aggressive cells lose invasion activity when exposed to SATB1 siRNA and non-tumorigenic cells gain invasive activity when SATB1 is overexpressed.

FIG. 14A-14C are comparison of SATB1-dependent genes with the prognosis signature genes. FIG. 14A Microarray data from MDA-MB-231-control shRNA/SATB1-shRNA were compared to data from van′t Veer L. J. et al. for prognostic signature genes. Among 231 poor prognostic signature genes, 174 were identified in our Codelink and Affymetrix GeneChip data. 36% (63/174 genes) of these genes up/downregulated during tumorigenesis were correspondingly regulated by SATB1 (P=0.02). FIG. 14B 103 bone metastasis marker genes were also found in our microarray analyses; 28 of these genes (shown, 27%) are correspondingly up/downregulated by SATB1 (P=0.0002). FIG. 14C SATB1-regulated genes were compared with lung-metastasis promoting signatures previously reported. 105 marker genes matched genes identified in our microarrays, and 25 genes (shown, 23%) were found to be correspondingly regulated by SATB1 (either up or downregulated) (P=0.021). Color codes at bottom indicate relative-fold expression levels.

Table 1 is a summary of pathological information of human primary breast tumor specimens which were used in this study.

Table 2 is a summary of data showing association between nuclear SATB1 score and clinicopathological characteristics.

### DETAILED DESCRIPTION OF THE PREFERRED EMBODIMENT

### Abbreviations

SATB1; Special AT-rich binding protein 1
HMEC; Human mammary epithelial cell lines
SATB1-siRNA or SATB1-shRNA; Short hairpin-interfering RNAs against SATB1
2D culture; Two dimensional culture, Plastic dish culture
3D culture; Three dimensional culture on matrigel
RT-PCR; Reverse Transcription-Polymerase chain reaction
GO; Gene ontology
EMT; Epithelial-mesenchymal transition
ChIP; Chromatin immunoprecipitation
LM-PCR; Ligation mediated polymerase chain reaction
IL-2Rα; Interleukin-2 receptor alphaα
BRMS1; Breast carcinoma metastasis suppressor 1
PLAUR; Plasminogen activator urokinase

### OB; Osteoblast

BM; basement membrane

The present invention provides methods and compositions based upon recent discovery by the inventors that indicate that a high level of SATB1 expression correlates with the ability of breast cancer cells to invade in vitro and metastasize in vivo. High clinical relevance of SATB1 was found in human breast cancer (shown in the examples). Among the 28 tumor samples, sixteen were metastatic breast carcinomas. SATB1 was expressed in all of 16 metastatic breast carcinoma samples with very high statistical significance (P<0.0001) compared to either moderately differentiated tumor or normal tissue samples. SATB1 was not detected in any normal adjacent tissues. SATB1 was also found to be expressed in small lung cell carcinoma, leukemia (in Jurkat cells, CEM cells), lymphomas and colon cancers (data not shown). Thus it is contemplated that SATB1 may be used in prognostic, diagnostic and therapeutic applications as described herein for these and other aggressive or advanced cancers.

While diagnosis and detection of other cancers may rely on gene amplification of certain genes, the present invention relies on the ectopic expression of SATB1. In addition, there might be an alternative form of SATB1 specific to cancer and this might represent a post-translationally modified version of SATB1 which is expressed in aggressive cancers. Because SATB1 is a gene regulator, SATB1 in its cancer-specific form is believed to turn on and organize genes involved in metastatic cancers and confer determinant roles in cell morphogenesis, cell motility, and the invasive activity of cancer cells in vivo.

Although detection of SATB1 within breast epithelial cells of breast tissue biopsy sample is sufficient to identify aggressive breast cancer cells, SATB1 may also be detected in activated lymphocytes. Therefore, a cancer-specific form of SATB1 would be a useful marker for specifically detecting malignant cells. For example, to biochemically characterize cancer specific SATB1, we can use BUR affinity chromatography to purify SATB1 from metastatic breast cancer specimen using the established method as described in Kohwi-Shigematsu et al., Methods in Cell Biology 53: 324-352, 1998.

Specific region of modification in a cancer-specific SATB1 protein can be identified by techniques known and useful in the art, such as nuclear magnetic resonance (NMR), MALDI analysis (e.g., MALDI-TOF). This is similar to a case for a cancer-specific protein, PCNA observed by ds as described or adapted from Bechtel P E, et al. who found “A unique form of proliferating cell nuclear antigen is present in malignant breast cells.” Cancer Res. 1998 August 1; 58(15):3264-9. Methods can be also be used or adapted as described by Naryzhny S N A and Lee H, “Observation of multiple isoforms and specific proteolysis patterns of proliferating cell nuclear antigen in the context of cell cycle compartments and sample preparations,” Proteomics. 2003 June; 3(6):930-6, which describes data consistent with the idea that the existence of the different isoforms and specific proteolysis of PCNA are relevant to its functions in vivo. Both references are hereby incorporated by reference in their entirety.

Therefore reagents and tools can be created by methods known in the art based upon and to detect SATB1 protein specifically expressed in aggressive breast cancer cells for use in diagnosis and prognosis. In another embodiment, therapeutics can be made to inhibit SATB1 protein to deplete its expression or block its function in metastatic and aggressive cancers.

A. Diagnostic and/or Prognostic Applications Using SATB1

In one embodiment of the invention, methods for detection of SATB1 protein is provided for use in diagnosis and prognosis of metastatic and aggressive cancers. In a specific embodiment, the cancer detected is breast cancer. In other embodiments, SATB1 is detected in cancers such as lung small lung cell carcinoma, leukemia (in Jurkat cells, CEM cells), lymphomas, bone and colon cancers. As stated above, such applications can be made because in normal tissues SATB1 was undetectable, and low to undetectable levels of SATB1 expression were detected in carcinoma originally diagnosed as moderately differentiated infiltrating ductal carcinomas and high levels of SATB1 expression can be detected in metastatic breast carcinomas.

1. Nucleotide Detection of SATB1

In one embodiment, a PCR assay is used to detect SATB1 expression. Primers can be created using the unique sequences of SATB1 (SEQ ID NO: 1) or the genomic sequence, to detect sequence amplification by signal amplification in gel electrophoresis. As is known in the art, primers or oligonucleotides are generally 15-40 bp in length, and usually flank unique sequence that can be amplified by methods such as polymerase chain reaction (PCR) or reverse transcriptase PCR. Primers to detect SATB1 expression can be created based upon genomic sequence containing and flanking SATB1. SATB1 is located on chromosome 3p23, GeneID 6304 and the Unigene Locus number is Hs.517717. Useful sequences for making probes and other sequences in the present invention include but are not limited, human SATB1 mRNA found at GenBank Accession No. NM 002971.2 (GI:33356175), and human SATB1 protein sequence, GenBank Accession No. NP_{—}002962, all of which are hereby incorporated by reference.

In a preferred embodiment, SATB1 expression is detected using an RT-PCR assay to detect SATB1 transcription levels in aggressive cancer cells.

In another embodiment, SATB1 expression is detected by colorimetric detection using a bio-barcode assay as described in Mirkin et al., U.S. Pat. Appln. Nos. 20020192687 and 20050037397 which describe bio-barcode based detection of target analytes.

2. Antibody Detection of SATB1

In another embodiment, ectopic SATB1 expression in aggressive breast cancer cells can be detected using an immunohistochemical assay of human biopsy tissue specimen. Anti-SATB1 antibodies can be made by general methods known in the art and as described in U.S. Pat. Nos. 5,652,340 and 5,869,621, both which are hereby incorporated by reference in their entirety for all purposes. Once the cancer-specific form of SATB1 is fully characterized, antibodies specific for this form can also be made. Such antibodies will greatly aid in detecting the specific form of SATB1 in aggressive cancer in a Western blot assay of whole tissue extracts, which may contain activated lymphocytes expressing the normal SATB1 protein. Antibodies against cancer specific SATB1 should be able to distinguish SATB1 expressed in aggressive breast cancer cells from that in activated lymphocytes using whole cell extracts prepared from biopsy samples.

Polyclonal and monoclonal antibodies can be made by well-known methods in the art. A preferred method of generating these antibodies is by first synthesizing peptide fragments from the SATB1 protein. These peptide fragments should likely cover unique regions in the SATB1 gene which are subject to altered post-translational modifications as compared to normal SATB1, such as peptides SEQ ID NO: 2 and SEQ ID NO: 3. If a specific type of modification is found in cancer-specific SATB1, a peptide with proper modification can be synthesized. Since synthesized peptides are not always immunogenic by their own, the peptides should be conjugated to a carrier protein before use. Appropriate carrier proteins include but are not limited to Keyhole limpet hemacyanin (KLH). The conjugated phospho peptides should then be mixed with adjuvant and injected into a mammal, preferably a rabbit through intradermal injection, to elicit an immunogenic response. Samples of serum can be collected and tested by ELISA assay to determine the titer of the antibodies and then harvested.

Polyclonal (e.g., anti-SATB1) antibodies can be purified by passing the harvested antibodies through an affinity column. Monoclonal antibodies are preferred over polyclonal antibodies and can be generated according to standard methods known in the art of creating an immortal cell line which expresses the antibody. In one embodiment, a SATB1 antibody as a control is an antibody of U.S. Pat. No. 5,869,621.

Nonhuman antibodies are highly immunogenic in human and that limits their therapeutic potential. In order to reduce their immunogenicity, nonhuman antibodies need to be humanized for therapeutic application. Through the years, many researchers have developed different strategies to humanize the nonhuman antibodies. One such example is using “HuMAb-Mouse” technology available from MEDAREX, Inc. and disclosed by van de Winkel, in U.S. Pat. No. 6,111,166 and hereby incorporated by reference in its entirety. “HuMAb-Mouse” is a strain of transgenic mice which harbor the entire human immunoglobin (Ig) loci and thus can be used to produce fully human monoclonal antibodies such as monoclonal anti-SATB1 antibodies.

In one embodiment, immunohistochemical analysis using an antibody against normal SATB1 will detect aggressive malignant breast cancer and activated T cells present in the tissue specimens which can be distinguished by cell shapes. However, immunohistochemical analysis of fixed tissue specimens and Western blot analysis of cell extracts using an antibody against cancer specific-SATB1 will specifically detect the presence of aggressive breast cancer cells which have the potential to metastasize in a given specimen.

In another embodiment, the anti-SATB1 antibodies are used to aid in the detection of other types of cancer including small lung cell carcinoma, leukemia (in Jurkat cells, CEM cells), lymphomas and colon cancers, and other advanced cancers.

B. Therapeutic Applications Using SATB1

SATB1 is a key molecule which affects the aggressiveness of breast cancer cells. Therefore, in another embodiment, we will manipulate expression of SATB1, preferably by depletion of active or functional SATB1. The invention further provides for compounds to treat malignant cells ectopically expressing SATB1. In a preferred embodiment, the compound is a SATB1 inhibitor such as, an antisense oligonucleotide; a siRNA/shRNA olignonucleotide; a small molecule that interferes with SATB1 function; a viral vector producing a nucleic acid sequence that inhibits SATB1; or an aptamer.

For example, such manipulation can be made using optimized shRNAs. Strong Pearson correlations between target expression levels and normalizing effects of shRNAs will indicate that expression levels determine the extent of response to target protein inhibitors. High throughput methods can be used to identify SATB1 inhibitors such as shRNA and/or small molecular inhibitor formulations to deliver SATB1 inhibitors efficiently to cultured cells and xenografts. Cancer-specific SATB1 inhibitory formulations will be preferentially effective against xenografts that have cancer-specific SATB1 expression and that these formulations will inhibit the formation, development or growth of cancer. Effective formulations using such methods as described herein will be developed for clinical application.

1. RNA Interference (RNAi) Sequence Design

In one embodiment, known methods are used to identify sequences that inhibit SATB1. Such inhibitors may include but are not limited to, siRNAoligonucleotides, antisense oligonucleotides, peptide inhibitors and aptamer sequences that bind and act to inhibit SATB1 expression and/or function. In another embodiment, inhibitor siRNA or antisense oligonucleotides are used specifically to target SATB1 and SATBlexpression.

In one embodiment, RNA interference is used to generate small double-stranded RNA (small interference RNA (siRNA) or short hairpin RNA (shRNA)) inhibitors to affect the expression of a candidate gene generally through cleaving and destroying its cognate RNA. Herein siRNA and shRNA may be used interchangeably. Small interference RNA (siRNA or shRNA) is typically 19-22 nt double-stranded RNA. siRNA can be obtained by chemical synthesis or by DNA-vector based RNAi technology. Using DNA vector based siRNA technology, a small DNA insert (about 70 bp) encoding a short hairpin RNA targeting the gene of interest is cloned into a commercially available vector. The insert-containing vector can be transfected into the cell, and expressing the short hairpin RNA. The hairpin RNA is rapidly processed by the cellular machinery into 19-22 nt double stranded RNA (siRNA). In a preferred embodiment, the siRNA is inserted into a suitable RNAi vector because siRNA made synthetically tends to be less stable and not as effective in transfection.

siRNA can be made using methods and algorithms such as those described by Wang L, Mu F Y. (2004) A Web-based Design Center for Vector-based siRNA and siRNA cassette. Bioinformatics. (In press); Khvorova A, Reynolds A, Jayasena S D. (2003) Functional siRNAs and miRNAs exhibit strand bias. Cell. 115(2):209-16; Harborth J, Elbashir S M, Vandenburgh K, Manninga H, Scaringe S A, Weber K, Tuschl T. (2003) Sequence, chemical, and structural variation of small interfering RNAs and short hairpin RNAs and the effect on mammalian gene silencing. Antisense Nucleic Acid Drug Dev. 13(2):83-105; Reynolds A, Leake D, Boese Q, Scaringe S, Marshall W S, Khvorova A. (2004) Rational siRNA design for RNA interference. Nat Biotechnol. 22(3):326-30 and Ui-Tei K, Naito Y, Takahashi F, Haraguchi T, Ohki-Hamazaki H, Juni A, Ueda R, Saigo K. (2004) Guidelines for the selection of highly effective siRNA sequences for mammalian and chick RNA interference. Nucleic Acids Res. 32(3):936-48, which are hereby incorporated by reference.

Other tools for constructing siRNA sequences are web tools such as the siRNA Target Finder and Construct Builder available from GenScript, Oligo Design and Analysis Tools from Integrated DNA Technologies, or siDESIGN™ Center from Dharmacon, Inc. siRNA are suggested to be built using the ORF (open reading frame) as the target selecting region, preferably 50-100 nt downstream of the start codon. Because siRNAs function at the mRNA level, not at the protein level, to design an siRNA, the precise target mRNA nucleotide sequence may be required. Due to the degenerate nature of the genetic code and codon bias, it is difficult to accurately predict the correct nucleotide sequence from the peptide sequence. Additionally, since the function of siRNAs is to cleave mRNA sequences, it is important to use the mRNA nucleotide sequence and not the genomic sequence for siRNA design, although as noted in the Examples, the genomic sequence can be successfully used for siRNA design. However, designs using genomic information might inadvertently target introns and as a result the siRNA would not be functional for silencing the corresponding mRNA.

Rational siRNA design should also minimize off-target effects which often arise from partial complementarity of the sense or antisense strands to an unintended target. These effects are known to have a concentration dependence and one way to minimize off-target effects is often by reducing siRNA concentrations. Another way to minimize such off-target effects is to screen the siRNA for target specificity.

In one embodiment, the siRNA can be modified on the 5′-end of the sense strand to present compounds such as fluorescent dyes, chemical groups, or polar groups. Modification at the 5′-end of the antisense strand has been shown to interfere with siRNA silencing activity and therefore this position is not recommended for modification. Modifications at the other three termini have been shown to have minimal to no effect on silencing activity.

It is recommended that primers be designed to bracket one of the siRNA cleavage sites as this will help eliminate possible bias in the data (i.e., one of the primers should be upstream of the cleavage site, the other should be downstream of the cleavage site). Bias may be introduced into the experiment if the PCR amplifies either 5′ or 3′ of a cleavage site, in part because it is difficult to anticipate how long the cleaved mRNA product may persist prior to being degraded. If the amplified region contains the cleavage site, then no amplification can occur if the siRNA has performed its function.

In a preferred embodiment, at least one sequence such as SEQ ID NO: 3 was used to design the SATB1 shRNA sequences SEQ ID NOs: 4-7. In a preferred embodiment, using SEQ ID NO: 3 as the target sequence and siRNA Target Finder from Ambion, Inc, siRNAs are designed that deplete SATB1 in malignant cells and return the cell phenotype to that of a non-invasive phenotype. In another preferred embodiment, the SATB1 shRNAs have the sequence of SEQ ID NO: 4 (sense) and SEQ ID NO: 5 (anti-sense) or SEQ ID NO: 6 (sense) and SEQ ID NO: 7 (anti-sense). The sequences of SEQ ID NOS: 4-7 are given below:

shRNA 1 sequence (SENSE)
SEQ ID NO: 4
GATCCCCGGATTTGGAAGAGAGTGTCTTCAAGAGAGACACTCTC
TTCCAAATCCTTTTTGGAAA
shRNA 1 sequence (Anti-SENSE)
SEQ ID NO: 5
AGCTTTTCCAAAAAGGATTTGGAAGAGAGTGTCTCTCTTGAAGA
CACTCTCTTCCAAATCCGGG
shRNA 2 sequence (SENSE)
SEQ ID NO: 6
GATCCCCGTCCACCTTGTCTTCTCTCTTCAAGAGAGAGAGAAGA
CAAGGTGGACTTTTTGGAAA
shRNA 2 sequence (Anti-SENSE)
SEQ ID NO: 7
AGCTTTTCCAAAAAGTCCACCTTGTCTTCTCTCTCTCTTGAAGA
GAGAAGACAAGGTGGACGGG

In another embodiment, a webdesigning tool from Genescript may be used since it provides the top candidates and also performs BLAST screening (Altschul, S. F., Gish, W., Miller, W., Myers, E. W. & Lipman, D. J. (1990) “Basic local alignment search tool.” J. Mol. Biol. 215:403-410) on each resulting siRNA sequence.

2. Inhibitor Antisense Oligonucleotide

In another embodiment, antisense oligonucleotides (“oligos” and “oligomers”) can be designed to inhibit SATB1 and other candidate gene function. Antisense oligonucleotides are short single-stranded nucleic acids, which function by selectively hybridizing to their target mRNA, thereby blocking translation. Translation is inhibited by either RNase H nuclease activity at the DNA:RNA duplex, or by inhibiting ribosome progression, thereby inhibiting protein synthesis. This results in discontinued synthesis and subsequent loss of function of the protein for which the target mRNA encodes.

In a preferred embodiment, antisense oligos are phosphorothioated upon synthesis and purification, and are usually 18-22 bases in length. It is contemplated that the SATB1 antisense oligos may have other modifications such as 2′-O-Methyl RNA, methylphosphonates, chimeric oligos, modified bases and many others modifications, including fluorescent oligos.

In a preferred embodiment, active antisense oligos should be compared against control oligos that have the same general chemistry, base composition, and length as the antisense oligo. These can include inverse sequences, scrambled sequences, and sense sequences. The inverse and scrambled are recommended because they have the same base composition, thus same molecular weight and Tm as the active antisense oligonucleotides. Rational antisense oligo design should consider, for example, that the antisense oligos do not anneal to an unintended mRNA or do not contain motifs known to invoke immunostimulatory responses such as four contiguous G residues, palindromes of 6 or more bases and CG motifs.

Antisense oligonucleotides can be used in vitro in most cell types with good results. However, some cell types require the use of transfection reagents to effect efficient transport into cellular interiors. It is recommended that optimization experiments be performed by using differing final oligonucleotide concentrations in the 1-5 μm range with in most cases the addition of transfection reagents. The window of opportunity, i.e., that concentration where you will obtain a reproducible antisense effect, may be quite narrow, where above that range you may experience confusing non-specific, non-antisense effects, and below that range you may not see any results at all. In a preferred embodiment, down regulation of the targeted mRNA (e.g., SATB1 mRNA SEQ ID NO: 1) will be demonstrated by use of techniques such as northern blot, real-time PCR, cDNA/oligo array or western blot. The same endpoints can be made for in vivo experiments, while also assessing behavioral endpoints.

For cell culture, antisense oligonucleotides should be re-suspended in sterile nuclease-free water (the use of DEPC-treated water is not recommended). Antisense oligonucleotides can be purified, lyophilized, and ready for use upon re-suspension. Upon suspension, antisense oligonucleotide stock solutions may be frozen at −20° C. and stable for several weeks.

3. High Throughput Screening for Small Molecule SATB1 Inhibitors

In one embodiment, high throughput screening (HTS) methods are used to identify compounds that inhibit SATB1. HTS methods involve providing a combinatorial chemical or peptide library containing a large number of potential therapeutic compounds (i.e., compounds that inhibit SATB1). Such “libraries” are then screened in one or more assays, as described herein, to identify those library members (particular peptides, chemical species or subclasses) that display the desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., ECIS™, Applied BioPhysics Inc., Troy, N.Y., MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

4. SATB1 Inhibitor Antibodies

In one embodiment, depletion of SATB1 will be made using inhibitors preferentially toxic to cells which are ectopically expressing SATB1. It is contemplated that such depletion will in turn decrease expression of genes which allow cancer cells to acquire metastasizing activity because of their crucial role in cell invasion, motility, altered morphology and anchorage-dependent growth. Thus, the depletion of SATB1 should ultimately prevent tumor formation and metastasis in aggressive cancers and decrease tumorigenicity.

In other embodiments, polyclonal or monoclonal antibodies that specifically bind or inhibit SATB1, can be used using methods known in the art and may be used therapeutically as well. In other embodiments, polyclonal or monoclonal antibodies that specifically bind or inhibit SATB1, can be used using methods known in the art and as described above. It is contemplated that the monoclonal antibodies may be used therapeutically as well. Such use of antibodies has been demonstrated by others and may be useful in the present invention to inhibit or downregulate SATB1.

5. Recombinant Expression, Synthesis and Isolation of SATB1 Inhibitors

SATB1 inhibitors such as the siRNA SATB1 inhibitor described herein can also be made using nucleic acid or peptide synthesis or expressed recombinantly. The entire inhibitor sequence can be made using commercial oligonucleotide synthesis or peptide synthesis. The invention further contemplates the use of both native and modified DNA and RNA bases, e.g. beta-D-Glucosyl-Hydroxymethyluracil, and native and modified amino acid residues.

In other embodiments, the nucleic acid sequences encoding SATB1 inhibitors such as the siRNA SATB1 inhibitor and related nucleic acid sequence homologues can be cloned. This aspect of the invention relies on routine techniques in the field of recombinant genetics. Generally, the nomenclature and the laboratory procedures in recombinant DNA technology described herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. Basic texts disclosing the general methods of use in this invention include Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 1994)).

Substantially identical nucleic acids encoding sequences of SATB1 inhibitors can be isolated using nucleic acid probes and oligonucleotides under stringent hybridization conditions, by screening libraries. Alternatively, expression libraries can be used to clone these sequences, by detecting expressed homologues immunologically with antisera or purified antibodies made against the core domain of nucleic acids encoding SATB1 inhibitor sequences.

Gene expression of SATB1 can also be analyzed by techniques known in the art, e.g reverse transcription and amplification of mRNA, isolation of total RNA or poly A+RNA, northern blotting, dot blotting, in situ hybridization, RNase protection, probing DNA microchip arrays, and the like.

To obtain high level expression of a cloned gene or nucleic acid sequence, such as those cDNAs encoding nucleic acid sequences encoding SATB1 inhibitors such as the shRNA SATB1 inhibitor and related nucleic acid sequence homologues, one typically subclones an inhibitor peptide sequence (e.g., nucleic acid sequences encoding SATB1 inhibitors such as the shRNA SATB1 inhibitor and related nucleic acid sequence homologue or a sequence encoding SEQ ID NOS:4-7) into an expression vector that is subsequently transfected into a suitable host cell. The expression vector typically contains a strong promoter or a promoter/enhancer to direct transcription, a transcription/translation terminator, and for a nucleic acid encoding a protein, a ribosome binding site for translational initiation. The promoter is operably linked to the nucleic acid sequence encoding SATB1 inhibitors such as the shRNA SATB1 inhibitor or a subsequence thereof. Suitable bacterial promoters are well known in the art and described, e.g., in Sambrook et al. and Ausubel et al. The elements that are typically included in expression vectors also include a replicon that functions in a suitable host cell such as E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to the recombinant SATB1 inhibitors peptides to provide convenient methods of isolation, e.g., His tags. In some case, enzymatic cleavage sequences (e.g., Met-(His)-Ile-Glu-Gly-Arg (SEQ ID NO:10) which form the Factor Xa cleavage site) are added to the recombinant SATB1 inhibitor peptides. Bacterial expression systems for expressing the SATB1 inhibitor peptides and nucleic acids are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Standard transfection methods are used to produce cell lines that express large quantities of SATB1 inhibitor, which can then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of cells is performed according to standard techniques (see, e.g., Morrison, J. Bact. 132:349-351 (1977); Clark-Curtiss & Curtiss, Methods in Enzymology 101:347-362 (Wu et al., eds, 1983). For example, any of the well known procedures for introducing foreign nucleotide sequences into host cells may be used. These include the use of calcium phosphate transfection, lipofectamine, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing SATB1 inhibitor peptides and nucleic acids.

After the expression vector is introduced into the cells, the transfected cells are cultured under conditions favoring expression of SATB1 inhibitors such as the siRNA SATB1 inhibitor and related nucleic acid sequence homologues.

6. Gene Therapy

In certain embodiments, the nucleic acids encoding inhibitory SATB1 peptides and nucleic acids of the present invention can be used for transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acid, under the control of a promoter, then expresses an inhibitory SATB1 peptides and nucleic acids of the present invention, thereby mitigating the effects of ectopic expression of SATB1 in malignant cells.

Such gene therapy procedures have been used to correct acquired and inherited genetic defects, cancer, and other diseases in a number of contexts. The ability to express artificial genes in humans facilitates the prevention and/or cure of many important human diseases, including many diseases which are not amenable to treatment by other therapies.

For delivery of nucleic acids, viral vectors may be used. Suitable vectors include, for example, herpes simplex virus vectors as described in Lilley et al., Curr. Gene Ther. 1(4):339-58 (2001), alphavirus DNA and particle replicons as decribed in e.g., Polo et al., Dev. Biol. (Basel) 104:181-5 (2000), Epstein-Barr virus (EBV)-based plasmid vectors as described in, e.g., Mazda, Curr. Gene Ther. 2(3):379-92 (2002), EBV replicon vector systems as described in e.g., Otomo et al., J. Gene Med. 3(4):345-52 (2001), adeno-virus associated viruses from rhesus monkeys as described in e.g., Gao et al., PNAS USA. 99(18):11854 (2002), adenoviral and adeno-associated viral vectors as described in, e.g., Nicklin and Baker, Curr. Gene Ther. 2(3):273-93 (2002). Other suitable adeno-associated virus (AAV) vector systems can be readily constructed using techniques well known in the art (see, e.g., U.S. Pat. Nos. 5,173,414 and 5,139,941; PCT Publication Nos. WO 92/01070 and WO 93/03769). Additional suitable vectors include E1B gene-attenuated replicating adenoviruses described in, e.g., Kim et al., Cancer Gene Ther.9(9):725-36 (2002) and nonreplicating adenovirus vectors described in e.g., Pascual et al., J. Immunol. 160(9):4465-72 (1998). Exemplary vectors can be constructed as disclosed by Okayama et al. (1983) Mol. Cell. Biol. 3:280.

Molecular conjugate vectors, such as the adenovirus chimeric vectors described in Michael et al. (1993) J. Biol. Chem. 268:6866-6869 and Wagner et al. (1992) Proc. Natl. Acad. Sci. USA 89:6099-6103, can also be used for gene delivery according to the methods of the invention.

In one illustrative embodiment, retroviruses provide a convenient and effective platform for gene delivery systems. A selected nucleotide sequence encoding an inhibitory SATB1 nucleic acid or polypeptide can be inserted into a vector and packaged in retroviral particles using techniques known in the art. The recombinant virus can then be isolated and delivered to a subject. Suitable vectors include lentiviral vectors as described in e.g., Scherr and Eder, Curr. Gene Ther. 2(1):45-55 (2002). Additional illustrative retroviral systems have been described (e.g., U.S. Pat. No. 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Curr. Opin. Genet. Develop. 3:102-109.

7. SATB1 Inhibitor Aptamer Sequence Design

In another embodiment, aptamer sequences which bind to specific RNA or DNA sequences can be made. As used herein, the terms “aptamer(s)” or “aptamer sequence(s)” are meant to refer to single stranded nucleic acids (RNA or DNA) whose distinct nucleotide sequence determines the folding of the molecule into a unique three dimensional structure. Aptamers comprising 15 to 120 nucleotides can be selected in vitro from a randomized pool of oligonucleotides (10¹⁴-10¹⁵ molecules). Any aptamers of the invention as described herein further contemplates the use of both native and modified DNA and RNA bases, such as beta-D-Glucosyl-Hydroxymethyluracil.

Aptamer sequences can be isolated through methods such as those disclosed in co-pending U.S. patent application Ser. No. 10/934,856, entitled, “Aptamers and Methods for their Invitro Selection and Uses Thereof,” which is hereby incorporated by reference.

It is contemplated that the sequences described herein may be varied to result in substantially homologous sequences which retain the same function as the original. As used herein, a polynucleotide or fragment thereof is “substantially homologous” (or “substantially similar”) to another if, when optimally aligned (with appropriate nucleotide insertions or deletions) with the other polynucleotide (or its complementary strand), using an alignment program such as BLASTN (Altschul, S. F., Gish, W., Miller, W., Myers, E. W. & Lipman, D. J. (1990) “Basic local alignment search tool.” J. Mol. Biol. 215:403-410), and there is nucleotide sequence identity in at least about 80%, preferably at least about 90%, and more preferably at least about 95-98% of the nucleotide bases.

Nucleic acids encoding sequences of SATB1 inhibitors can also be isolated from expression libraries using antibodies as probes. Such polyclonal or monoclonal antibodies can be raised using, for example, the polypeptides comprising the sequences set forth in SEQ ID NOS: 5-8, and subsequences thereof, using methods known in the art (see, e.g., Harlow and Lane, Antibodies: A Laboratory Manual (1988).

8. Methods of Administration and Treatment

The SATB1 inhibitors of the present invention, such as the siRNA SATB1 inhibitor, also can be used to treat or prevent a variety of disorders associated with cancer. The antibodies, peptides and nucleic acids are administered to a patient in an amount sufficient to elicit a therapeutic response in the patient (e.g., inhibiting the development, growth or metastasis of cancerous cells; reduction of tumor size and growth rate, prolonged survival rate, reduction in concurrent cancer therapeutics administered to patient). An amount adequate to accomplish this is defined as “therapeutically effective dose or amount.”

The antibodies, peptides and nucleic acids of the invention can be administered directly to a mammalian subject using any route known in the art, including e.g., by injection (e.g., intravenous, intraperitoneal, subcutaneous, intramuscular, or intradermal), inhalation, transdermal application, rectal administration, or oral administration.

In other embodiments, such antibodies that specifically bind or inhibit SATB1, may be used therapeutically. Such use of antibodies has been demonstrated by others and may be useful in the present invention to inhibit or downregulate SATB1.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Administration of the antibodies, peptides and nucleic acids of the invention can be in any convenient manner, e.g., by injection, intratumoral injection, intravenous and arterial stents (including eluting stents), cather, oral administration, inhalation, transdermal application, or rectal administration. In some cases, the peptides and nucleic acids are formulated with a pharmaceutically acceptable carrier prior to administration. Pharmaceutically acceptable carriers are determined in part by the particular composition being administered (e.g., nucleic acid or polypeptide), as well as by the particular method used to administer the composition. Accordingly, there are a wide variety of suitable formulations of pharmaceutical compositions of the present invention (see, e.g., Remington's Pharmaceutical Sciences, 17th ed., 1989).

The present SATB1 inhibitors may be administered singly or in combination, and may further be administered in combination with other anti-neoplastic drugs known and determined by those familiar with the art. They may be conventionally prepared with excipients and stabilizers in sterilized, lyophilized powdered form for injection, or prepared with stabilizers and peptidase inhibitors of oral and gastrointestinal metabolism for oral administration.

The pharmaceutical forms suitable for injectable use include sterile aqueous solutions or dispersions and sterile powders for the extemporaneous preparation of sterile injectable solutions or dispersions (U.S. Pat. No. 5,466,468). The carrier can be a solvent or dispersion medium containing, for example, water, ethanol, polyol (e.g., glycerol, propylene glycol, and liquid polyethylene glycol, and the like), suitable mixtures thereof, and/or vegetable oils.

The dose administered to a patient, in the context of the present invention should be sufficient to effect a beneficial therapeutic response in the patient over time. The dose will be determined by the efficacy of the particular vector (e.g. peptide or nucleic acid) employed and the condition of the patient, as well as the body weight or surface area of the patient to be treated. The size of the dose also will be determined by the existence, nature, and extent of any adverse side-effects that accompany the administration of a particular peptide or nucleic acid in a particular patient.

In determining the effective amount of the vector to be administered in the treatment or prophylaxis of diseases or disorder associated with the disease, the physician evaluates circulating plasma levels of the polypeptide or nucleic acid, polypeptide or nucleic acid toxicities, progression of the disease (e.g., ovarian cancer), and the production of antibodies that specifically bind to the peptide. Typically, the dose equivalent of a polypeptide is from about 0.1 to about 50 mg per kg, preferably from about 1 to about 25 mg per kg, most preferably from about 1 to about 20 mg per kg body weight. In general, the dose equivalent of a naked c acid is from about 1 μg to about 100 μg for a typical 70 kilogram patient, and doses of vectors which include a viral particle are calculated to yield an equivalent amount of therapeutic nucleic acid.

For administration, antibodies, polypeptides and nucleic acids of the present invention can be administered at a rate determined by the LD-50 of the polypeptide or nucleic acid, and the side-effects of the antibody, polypeptide or nucleic acid at various concentrations, as applied to the mass and overall health of the patient. Administration can be accomplished via single or divided doses, e.g., doses administered on a regular basis (e.g., daily) for a period of time (e.g., 2, 3, 4, 5, 6, days or 1-3 weeks or more).

In certain circumstances it will be desirable to deliver the pharmaceutical compositions comprising the SATB1 inhibitor antibodies, peptides and nucleic acids of the present invention parenterally, intravenously, intramuscularly, or even intraperitoneally as described in U.S. Pat. No. 5,543,158; U.S. Pat. No. 5,641,515 and U.S. Pat. No. 5,399,363. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

In another embodiment, 5-20 micrograms of the present siRNA or antisense oligonucleotides can be suspended in 100 microliters of buffer such as PBS (phosphate buffered saline) for injecting into a subject intravenously to induce apoptosis of cancer cells. (See Slaton, Unger, Sloper, Davis, Ahmed, Induction of apoptosis by antisense CK2 in human prostate cancer xenograft model, Mol Cancer Res. 2004 December; 2(12):712-21.)

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

To date, most siRNA studies have been performed with siRNA formulated in sterile saline or phosphate buffered saline (PBS) that has ionic character similar to serum. There are minor differences in PBS compositions (with or without calcium, magnesium, etc.) and investigators should select a formulation best suited to the injection route and animal employed for the study. Lyophilized oligonucleotides and standard or stable siRNAs are readily soluble in aqueous solution and can be resuspended at concentrations as high as 2.0 mM. However, viscosity of the resultant solutions can sometimes affect the handling of such concentrated solutions.

9. Delivery of Therapeutics

In certain embodiments, the use of liposomes, nanocapsules, microparticles, microspheres, lipid particles, vesicles, and the like, are contemplated for the administration of the SATB1 inhibitory nucleic acids of the present invention. In particular, the compositions of the present invention may be formulated for delivery either encapsulated in or operatively attached to a lipid particle, a liposome, a vesicle, a nanosphere, or a nanoparticle or the like.

The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

Targeting is generally not a limitation in terms of the present invention. However, should specific targeting be desired, methods are available for this to be accomplished. For example, antibodies may be used to bind to the liposome surface and to direct the liposomes and its contents to particular cell types. Carbohydrate determinants (glycoprotein or glycolipid cell-surface components that play a role in cell-cell recognition, interaction and adhesion) may also be used as recognition sites as they have potential in directing liposomes to particular cell types.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use in the present invention. Such particles may be easily made, as described (Couvreur et al., 1980; 1988; zur Muhlen et al., 1998; Zambaux et al. 1998; Pinto-Alphandry et al., 1995 and U.S. Pat. No. 5,145,684). Others have described nanoparticles in U.S. Pat. Nos. 6,602,932; 6,071,533.

It is further contemplated that the SATB1 inhibitors of the present invention is delivered to cancerous cells in a subject using other microparticles, nanostructures and nanodevices. For example, microspheres may be used such as those available from PolyMicrospheres, Inc. (Indianapolis, Ind.). For descriptions of drug delivery, see generally Alivisatos A P, Less is more in medicine, Understanding Nanotechnology, Warner Books, New York, 2002; Max Sherman, The World of Nanotechnology, US Pharm. 2004; 12:HS-3-HS-4; Brannon-Peppas and Blanchette, Nanoparticle and targeted systems for cancer therapy, Advanced Drug Delivery Reviews, Intelligent Therapeutics: Biomimetic Systems and Nanotechnology in Drug Delivery, Volume 56, Issue 11, 22 Sep. 2004, Pages 1649-1659; and D. M. Brown, ed., Drug Delivery Systems in Cancer Therapy, Humana Press, Inc., Totowa, N.J. 2004, including Chapter 6: Microparticle Drug Delivery Systems by Birnbaum and Brannon-Peppas, pp. 117-136, all of which are hereby incorporated by reference.

10. Combination Therapy

In some embodiments, the inhibitory SATB1 nucleic acids are administered in combination with a second therapeutic agent for treating or preventing cancer. In one embodiment, an inhibitory SATB1 siRNA may be administered in conjunction with a second therapeutic agent for treating or preventing cancer. For example, an inhibitory SATB1 siRNA of SEQ ID NO: 3 and 4 or SATB1 shRNA SEQ ID NO: 8 and SEQ ID NO: 9, may be administered in conjunction with any of the standard treatments for cancer including, but not limited to, paclitaxel, cisplatin, carboplatin, chemotherapy, and radiation treatment.

The inhibitory SATB1 nucleic acids and the second therapeutic agent may be administered simultaneously or sequentially. For example, the inhibitory SATB1 nucleic acids may be administered first, followed by the second therapeutic agent. Alternatively, the second therapeutic agent may be administered first, followed by the inhibitory SATB1 nucleic acids. In some cases, the inhibitory SATB1 nucleic acids and the second therapeutic agent are administered in the same formulation. In other cases the inhibitory SATB1 nucleic acids and the second therapeutic agent are administered in different formulations. When the inhibitory SATB1 nucleic acids and the second therapeutic agent are administered in different formulations, their administration may be simultaneous or sequential.

In some cases, the inhibitory SATB1 nucleic acids can be used to target therapeutic agents to cells and tissues expressing SATB1 that are related to aggressive or advanced cancers. SATB1 has been found to be expressed in aggressive or advanced carcinomas, sarcomas, small lung cell carcinoma, leukemia (in Jurket cells), lymphomas, colon and breast cancers. Thus it is contemplated that the present invention may be used in the diagnostic and therapeutic applications described herein for these and other aggressive or advanced cancers.

11. Kits

Diagnostic/prognostic kit: The present invention further provides kits for use within any of the above diagnostic/prognostic methods. Such kits typically comprise two or more components necessary for performing a diagnostic/prognostic assay. Components of the kit may be compounds, reagents, containers and/or equipment. For example, one container within a kit may contain an antibody against SATB1 (normal version) and an antibody against cancer-specific SATB1. The kit contains buffers to dilute SATB1 antibodies, fluorescent dye-conjugated secondary antibodies (anti-mouse or anti-rabbit) to detect SATB1 signals. One or more additional containers may enclose elements, such as reagents or buffers, to be used in the assay. Such kits may also, or alternatively, contain a detection reagent as described above that contains a reporter group suitable for direct or indirect detection of antibody binding.

Kits for therapeutic uses. Thus, the subject compositions of the present invention may be provided, usually in a lyophilized form, in a container. The inhibitory SATB1 antibodies, chemicals, and/or nucleic acids described herein are included in the kits with instructions for use, and optionally with buffers, stabilizers, biocides, and inert proteins. Generally, these optional materials will be present at less than about 5% by weight, based on the amount of polypeptide or nucleic acid, and will usually be present in a total amount of at least about 0.001% by weight, based on the polypeptide or nucleic acid concentration. It may be desirable to include an inert extender or excipient to dilute the active ingredients, where the excipient may be present in from about 1 to 99% weight of the total composition. The kits may further comprise a second therapeutic agent, e.g., paclitaxel, carboplatin, or other chemotherapeutic agent.

### EXAMPLES

### Example 1

### SATB1 is Expressed in Highly Aggressive Cancer Cell Lines and Advanced Stages of Primary Tumor Samples, but not in Benign and Normal Samples

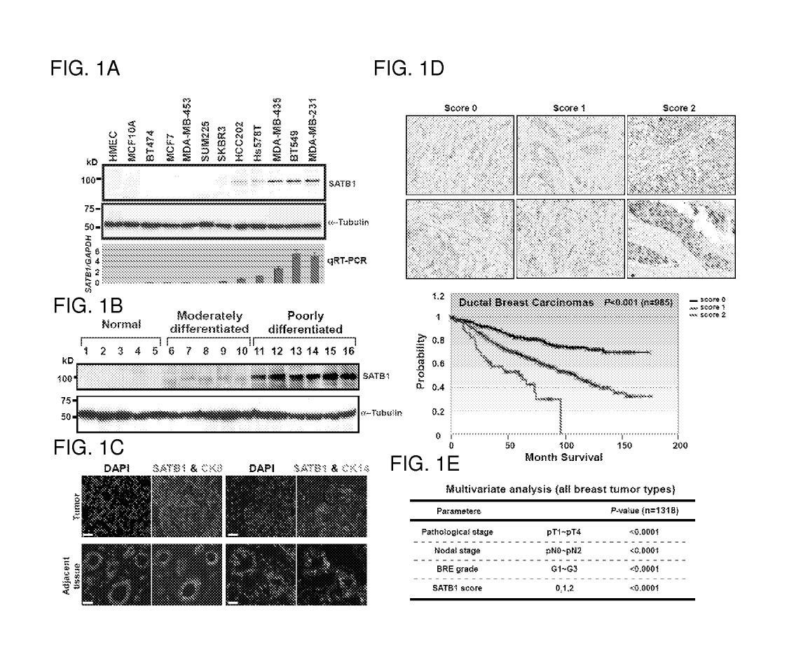

SATB1 expression levels was examined in 24 breast epithelial cell lines, including normal human mammary epithelial cells (HMEC) and 5 immortalized derivatives, 13 non-metastatic and 5 metastatic cancer cell lines. Both SATB1 mRNA and protein were detected only in metastatic cancer cell lines, correlating SATB1 expression with aggressive tumor phenotypes (results from representative cell lines shown in FIG. 1a). SATB2, a close homolog of SATB1, was expressed in both malignant and non-malignant cell lines (data not shown.

As shown in Table 1 and exemplified in FIG. 1B, SATB1 expression levels were examined in 28 human primary breast tumor samples, including moderately (12 cases) or poorly differentiated (16 cases) ductal carcinomas, and 10 adjacent tissues as controls. The pathological analyses for these tumor samples were made prior to our SATB1 expression analysis. High levels of SATB1 expression were detected in all lymph-node positive, poorly differentiated infiltrating ductal carcinomas, and low-level expression in some, but not all, moderately differentiated tumor samples (FIG. 1b and Table 1). SATB1 protein was detected in 23 of the 28 tumor samples examined. Among the 28 tumor samples, sixteen were metastatic breast carcinomas, and SATB1 was expressed in all of them with very high statistical significance (P<0.0001) compared to either moderately differentiated tumor or normal tissue samples. SATB1 was not detected in any normal adjacent tissues (FIG. 1b).

Representative immunostaining images of SATB1 and epithelial cell markers in invasive ductal carcinomas are shown in FIG. 1c Immunohistochemical analysis revealed that SATB1 expression was restricted to cells in cancerous areas of tissue samples, primarily in regions of highly disorganized morphology (FIG. 1c). DNA was counterstained with DAPI. Tissues structures were examined by hematoxylin and eosin (H& E).

SATB1 expression could be detected in aggressive breast carcinoma regardless of their classification ‘category’ based on available marker expression (Table 1). Table 1 shows the summary of pathological information of human primary breast tumor specimens which were used in this study.

SATB1's prognostic significance was determined by assessing its nuclear staining using tissue microarrays containing 2197 cases with known clinical follow-up records, from which 1318 breast cancer specimens were analyzable (Table 2 shows tumor composition and SATB1 association with clinico-pathological parameters). Tissues were scored based on the intensity of SATB1 nuclear labeling and percentage of SATB1-positive tumor cells. Immunohistochemistry was performed using a peroxidase detection system with human breast cancer tissue microarray slides (TriStar). Rabbit polyclonal SATB1 antibody (1583) was pre-absorbed against SKBR3 cell lysates fixed on activated PVDF membrane, applied (1:1800) and the slides were incubated overnight at 4° C. The slides were then counterstained with hematoxylin and mounted with permount (Fisher). To evaluate SATB1 levels, immunostained slides were scored with digital images obtained by ScanScope XT system (Aperio). The signal was scored based on the intensity and percentage of cells with SATB1 nuclear staining on the following scale: score 0, negative nuclear staining for all tumor cells; score 1, weak nuclear staining representing all the rest of score 0 and 2; score 2, moderate nuclear staining >50% or strong nuclear staining in >5% of the tumor cells. Samples that could not be interpreted or were missing most of the tumor tissue were given a score of not applicable (N/A). Scoring of the tissue microarray was completed by three independent observers. Significance of correlation between SATB1 signal and histopathological factors was determined using Pearson's Chi-squared (χ²) test. Kaplan-Meier plots were used to estimate the prognostic relevance of SATB1 in univariate analysis using WinStat (Fitch Software). Multivariate analysis was performed applying COX Proportional Hazards test.

Among these specimens, Kaplan-Meier survival analysis of 985 ductal carcinoma specimens revealed a correlation between higher SATB1 expression levels and shorter overall survival times (P<0.001) (FIG. 1d). This correlation was also observed with all breast cancer types (1318 specimens), except medullary cancer, which is rare and often has a relatively favorable prognosis despite its poorly-differentiated nuclear grade.

To exclude the possibility that the prognostic effect of nuclear SATB1 expression was dependent on other established prognostic factors for breast cancer, including tumor stage, BRE grade and nodal stage, we performed a multivariate analysis. This analysis confirmed that SATB1 is an independent prognostic factor for breast cancer (FIG. 1e).

A list of genes was compiled whose expression is SATB1 dependent in 2D or 3D culture conditions categorized in terms of biological pathway. Analysis of functional profile heterogeneity within the genes regulated by SATB1 was performed using web-based analysis tool, Pathway-express (URL: <http://vortex.cs.wayne.edu>). Among the 1200 genes that are either up or down-regulated by at least 2 fold in an SATB1-dependent manner in MDA-MB-231 cells, a total of 354 up-regulated genes and 267 down regulated genes that were involved in representative biological pathways based on KEGG (Kyoto Encyclopedia of genes and genomes) database were found. Genes whose expression was altered depending on SATB1 are mainly involved in the pathways of cell cycle, adherens junction, cytokine-receptor interaction, intracellular signaling, apoptosis and MAP kinase signaling.

### Example 2

### Construction of SATB1 Knocked-Down System

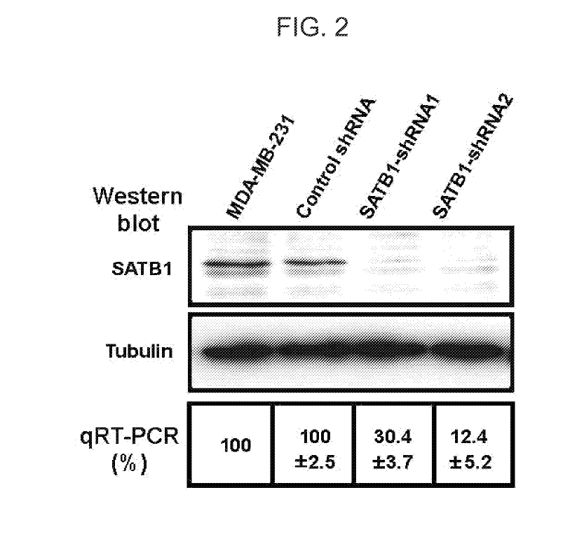

It was then tested whether suppression of SATB1 level in the highly metastatic MDA-MB-231 cells would affect their aggressiveness. Short hairpin-interfering RNAs (shRNA) were successfully designed to target SATB1 expression and are identified herein as SEQ ID NOs: 4-7. It was demonstrated that these shRNAs could suppress SATB1 expression in MDA-MB-231 cells by establishing cell clones stably transfected with pSUPER-puro (gift of Dr. Mina Bissell) bearing a DNA segment specifying such shRNA sequences (FIG. 2).

Two representative clones in which shRNAs drastically reduced the expression of SATB1 in protein and RNA level as well (FIG. 2) are shown. An shRNA, whose sequence did not match any known human gene, was also introduced into MDA-MB-231 cells. This control shRNA did not reduce SATB1 expression.

Two shRNAs were designed according to SATB1 sequence (GenBank Accession No. NM_{—}002971, hereby incorporated by reference) using siRNA Target Finder (Ambion, Austin, Tex.); The sequences of each oligoduplex were targeted as follows: shRNA₂₄₂₃, 5′-GGATTTGGAAGAGAGTGTC-3′ (SEQ ID NO: 8), or shRNA₂₅₉₅, 5′-GTCCACCTTGTCTTCTCTC-3′ (SEQ ID NO: 9). The oligoduplexes were cloned into the plasmid pSUPER (Oligoengine, Seattle, Wash.). Prepared DNAs were transfected into the aggressive breast cancer cell line MDA-MB-231 by lipofectamine 2000 (Invitrogen) and successfully transfected cells were selected by puromycin at 2 μg/ml or G418 at 1.5 mg/ml from 24 h after transfection. Single MDA-MB-231 cell clone stably expressing either shRNA₂₄₂₃ or shRNA₂₅₉₅ is designated SATB1-shRNA1 MDA or SATB1-shRNA2 MDA cells, respectively. For overexpression of SATB1, full length of SATB1 including 3′ UTR was cloned into retroviral vector pLXSN (Clontech, Mountain View, Calif.), and the viral solution was produced using PT67 package cell lines. Hs578T cells were infected with this viral solution, and stably infected cells were selected by G418 at 0.8 mg/ml for 5 days. The status of SATB1 level in manipulated MDA-MB-231 and Hs578T cells were examined by Western blot and real-time RT-PCR.

To evaluate the change of gene expression level depending on SATB1 status, semi-quantitative or real-time RT-PCR analysis of selected genes was performed (primer sequences are available upon request). Total RNA was extracted for cell lines using TRI reagent (Sigma) followed by RNA clean-up with RNeasy Mini kit (Qiagen, Valencia, Calif.). For semi-quantitative RT-PCR, 5 μg of total RNA was reverse-transcribed into single stranded cDNA using Superscript II RNaseH-reverse transcriptase (Invitrogen) according to the protocol supplied with the kit. PCR cycle was controlled starting from 25 to 40 cycles upon gene-specific primers (20 ng of cDNA/reaction). Each cycle consisted of the following steps, using GeneAmp PCR system 9700 (PerkinElmer Inc., Fremont, Calif.); 94° C. for 30 s, 55° C. for 30 s, and 72° C. for 60 s. PCR products were separated on 1.5% agarose gels and visualized them by staining with ethidium bromide. SYBR Green PCR Core Reagents system was used for real-time monitoring of amplification on ABI 7500 Fast Real-time PCR System (Applied Biosystems, Foster City, Calif.). Absolute quantification method was employed to quantify target DNA fragments in triplicate with following cycling condition; 95° C. for 2 min, followed by 40 cycles of 95° C. for 3 s and 60° C. for 30 s.

RNA interference (RNAi) was then used to determine whether SATB1 is required for the invasive and metastatic phenotypes of breast cancer cells. The highly-metastatic MDA-MB-231 cell line, derived from the pleural effusion of a breast cancer patient who developed widespread metastases years after removal of her primary tumor, expressed high levels of SATB1. Expressing short hairpin-interfering RNAs (shRNA) targeted against two SATB1 sequences in this cell line reduced its expression dramatically. Expression of SATB1 was lowered by 70% and 90%, respectively, in two transduced cell lines, which we named SATB1-shRNA1 MDA and SATB1-shRNA2 MDA cells (FIG. 2). SATB1 expression levels were 30.4%±3.7 in SATB1-shRNA1 MDA cells expressing SATB1-shRNA1 and only 12.5%±5.2 in SATB1-shRNA2 MDA cells expressing SATB1-shRNA2. SATB1 expression remained unaltered in MDA-MB-231 cells expressing an shRNA whose sequence did not match any known human gene (control cells).

### Example 3

### Depletion of SATB1 from Aggressive Breast Cancer Cells by shRNA 1) Reduced the Proliferation Rate, 2) Changed Cell Morphology, 3) Reversed the Invasive to Non-Invasive Phenotype and 4) LED to Loss of Anchorage-Independent Growth

Next we examined the effects of reduction of SATB1 expression in MDA-MB-231 cells in vitro on both the 2D (plastic) and on 3D (Matrigel) culture system, compared with the host MDA-MB-231 cells or those harboring control shRNA.

Reduction of Cell Proliferation Rate.

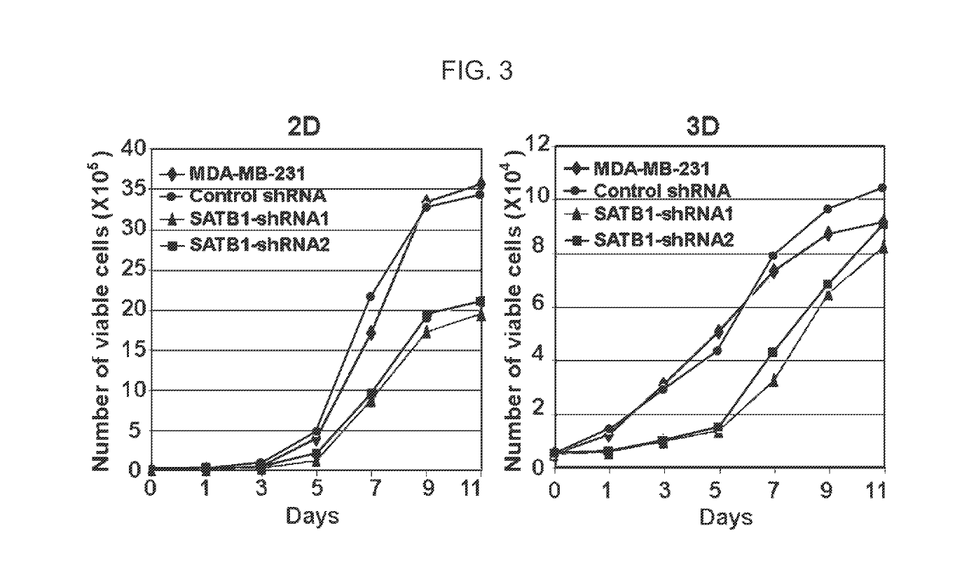

We examined whether loss of SATB1 expression affects cancer cell proliferation by culturing SATB1-shRNA1 MDA and SATB1-shRNA2 MDA cells on plastic dish {two-dimensional (2D) culture} or on a reconstituted basement membrane derived from Engelbreth-Holm-Swarm Tumor (Matrigel™) {three-dimensional (3D) culture}. The proliferation rates of SATB1-shRNA1 and SATB1-shRNA2 MDA cells were significantly reduced in both 2D and 3D cultures up to 11 days in culture, compared with their parental cell line and control cells (FIG. 3).

Cell Growth Assay. Total 2×10⁴ cells were plated on plastic dishes, and cultured for up to 10 days at 37° C. in 6-well plates (2D culture). Growth medium were renewed every 4 days. Cells were harvested by trypsin treatment, and counted at each time point using a cell counter (Beckman Coulter, Inc.; Fullerton, Calif.) and haematocytometer. Total 5×10³ cells were seeded on Matrigel (BD Biosciences, Inc.; Bedford, Mass.) coated 24-well plates (3D culture) as triplicate and incubated for up to 10 days at 37° C. Cells were treated with dispase (BD Biosciences, Inc.) for 2 h at 37° C. to be isolated from Matrigel, incubated with trypsin for further 5 min, and counted using a haematocytometer. Samples were analyzed in triplicate at 0, 2, 4, 6, 8, and 10 days after cell culture was initiated. Trypan blue exclusion analysis indicated that 99-100% of the cells were viable.

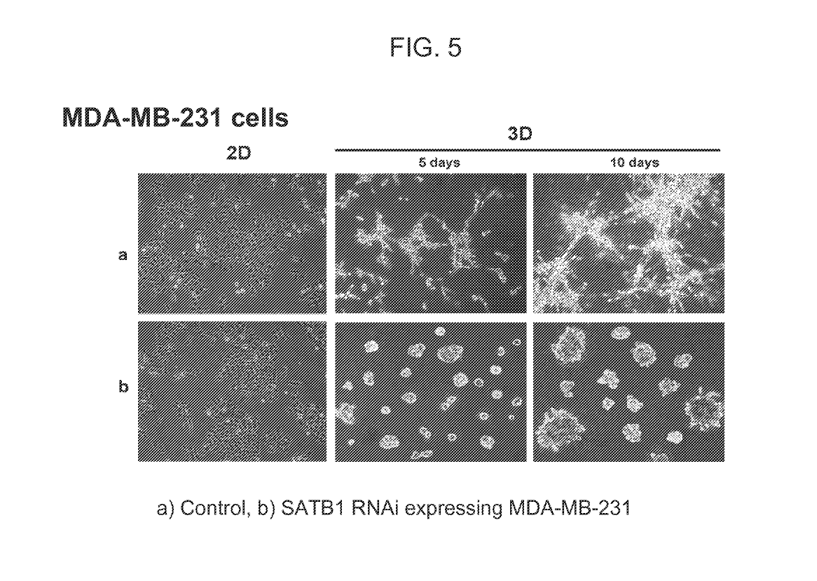

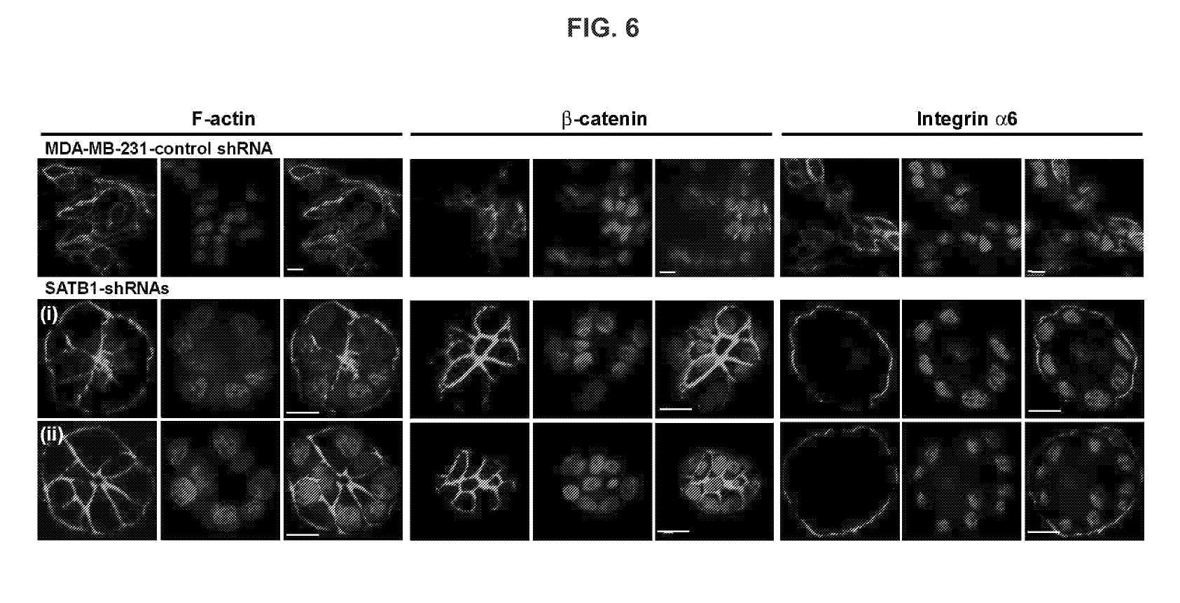

Changes in cell morphology. Referring now to FIG. 5, phase contrast micrographs of MDA-MB-231 control cells (control shRNA) or SATB1-shRNA1 MDA cells (SATB1-shRNA1) cultured on plastic (2D) and matrigel (3D) are shown. Major differences in cell morphology were observed when SATB1-shRNA1 MDA cells were grown in 3D culture, compared to control cells. The cell morphology was changed from stellate-like scattered (MDA-MB-231 with control shRNA) to spherical structures (MDA-MB-231 with SATB1-shRNA). Referring now to FIG. 6, immunostaining with antibodies against F-actin, β-catenin, integrin α6 (white), and counterstained with DAPI (DNA, faint white) indicated that SATB1-shRNA1 or SATB1-shRNA2 MDA cells grown in 3D culture had an organized and polarized morphology, forming acinus-like structures. In contrast, control cells in 3D lost polarity and showed disorganized morphology with altered lateral and basement membrane structures. As shown by immunostaining of actin and integrin α6, SATB1-shRNA harboring cells exhibited change in morphogenesis and formed acini within 3 days, while control cells formed large, loosely disorganized invasive colonies of cells. In addition, MDA-MB-231 cells expressing SATB1-shRNA were able to basally deposit and organize a basement membrane on Matrigel, showing the characteristic acini formation, even though there was a minor difference in morphology as observed in MCF10A control cells.

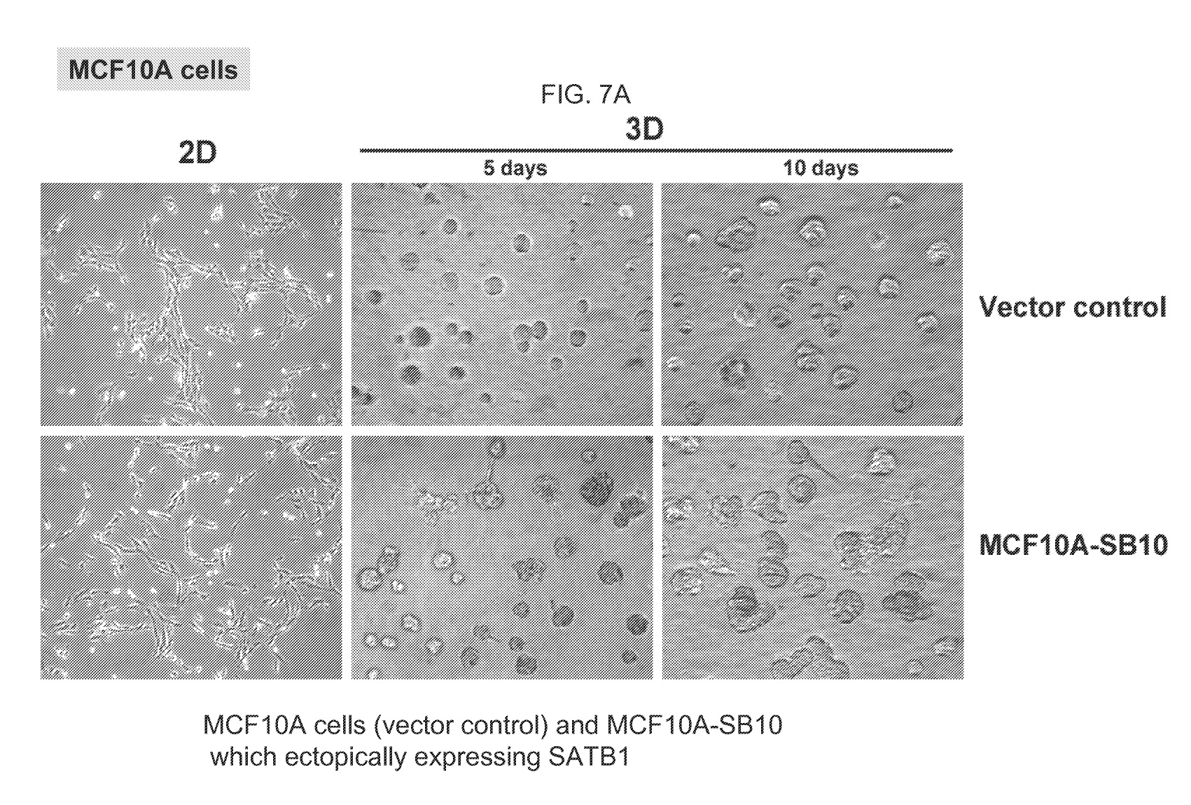

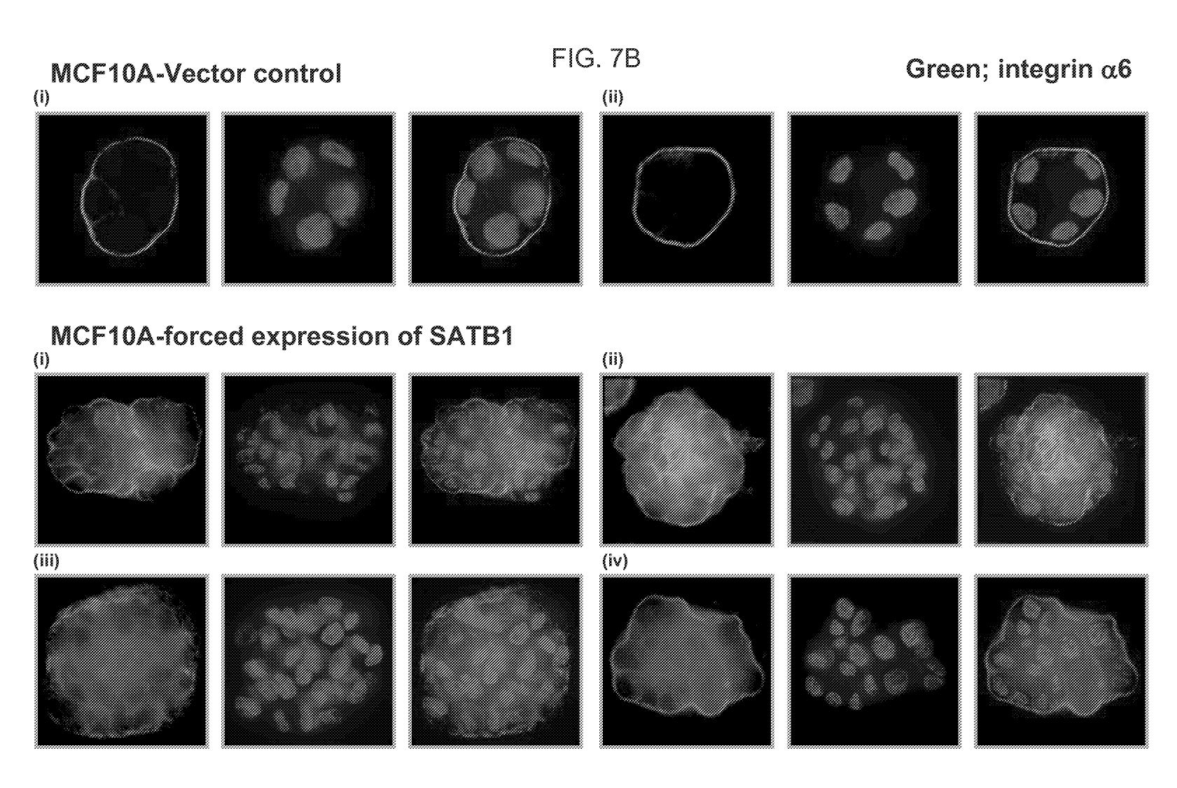

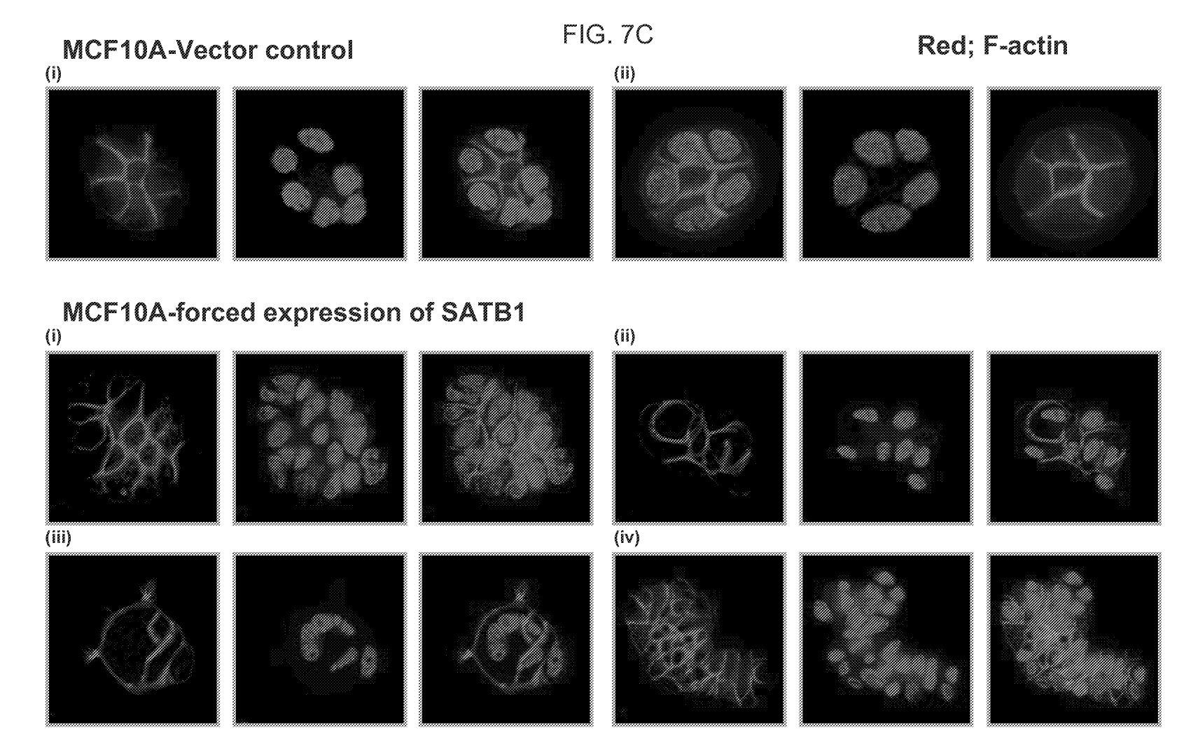

Referring to FIG. 7A, B, C, when SATB1 is forced to be expressed in non-tumorigenic MCF-10A cells, the normal acni structure changes to a disorganized structure where many cells grow on top of one another, losing cell polarity (see below).

The transition from a nonmalignant to a malignant cell is governed by mechanisms similar to those implicated in normal cellular differentiation and development. Epithelial cells might lose their polarity and adhesive contacts to become invasive carcinoma cells. Such a complex transformation has been summarized in the term epithelial-mesenchymal transition (EMT). Whether ectopic expression of SATB1 in MCF-10A promotes EMT is evaluated using stably transfected SATB1-overexpressing cell lines, by examining cell morphology on 3D culture on Matrigel. The morphological change from the cobble-stone-like appearance of epithelial cells to a spindle-like fibroblastic morphology is one of the hallmarks of an EMT. Examination of morphology visually can be by phase contrast microscopy, as well as by immunostaining. For cytoarchitecture, one can visualize actin filament and cytokeratin 18 distribution, and for polarity, examine collagen V, laminin and integrin distributions. One can also immunostain cells with markers such as E-cadherin, beta-catenin (epithelial markers), vimentin, fibronectin, and N-cadherin (fibroblast markers). We expect that SATB1 overexpressing MCF-10A cells would undergo EMT. These cells are expected to completely lose E-cadherin and beta-catenin, (markers for epithelial cells) and show expression of fibroblast markers, which correlate positively with the EMT. On the other hand, SATB1-depleted MDA-MB-231 cells would reverse EMT, and we expect that epithelial markers would be expressed and fibroblast markers would be lost.

Immunostaining of Cells in Culture.

Total 1×10⁴ cells mixed with Matrigel (5 mg/mL) were plated in 6-well plates pre-coated in a thick layer (1 mm) of Matrigel. Solidified Matrigel was overlaid with Growth medium (DMEM), and renewed at every 4 days. Colony formation and cell morphology was observed under light microscopy after 3 days to 14 days. Cells were fixed in 4% paraformaldehyde, permeablized in 0.1% Triton X100, and blocked in 5% BSA. Staining for SATB1 and focal adhesion compexes was performed by incubating with anti-SATB1, anti-β-catenin (clone 14), anti-integrin α6 (CD49f, all from BD Biosciences) antibodies, and fluorescein phalloidin for F-actin staining (Invitrogen Molecular Probe) for overnight at 4° C. For immunohistochemical analysis, breast carcinoma Tissue Microarray was obtained from US BioMax (Rockville, Md.). After deparaffinization, each slide was boiled in antigen unmasking solution (Vector Laboratories, Burlingame, Calif.) for 20 min. Tissue sections were incubated in 2% BSA/5% normal goat serum/0.1% Triton X-100 for 1 hour, and then subsequently reacted with primary antibodies against SATB1 (BD Biosciences) in blocking buffer for overnight at 4° C. Staining was detected with secondary Alexa Fluor 488 and/or Alexa Fluor 594 Abs (Molecular Probes). Cells were mounted in fluorescent mounting medium containing DAPI (Vector Laboratories). Images were collected by a DeltaVision microscope according to the manufacturer's instruction and processed with SoftWoRx software (Applied Precision, Issaquah, Wash.).

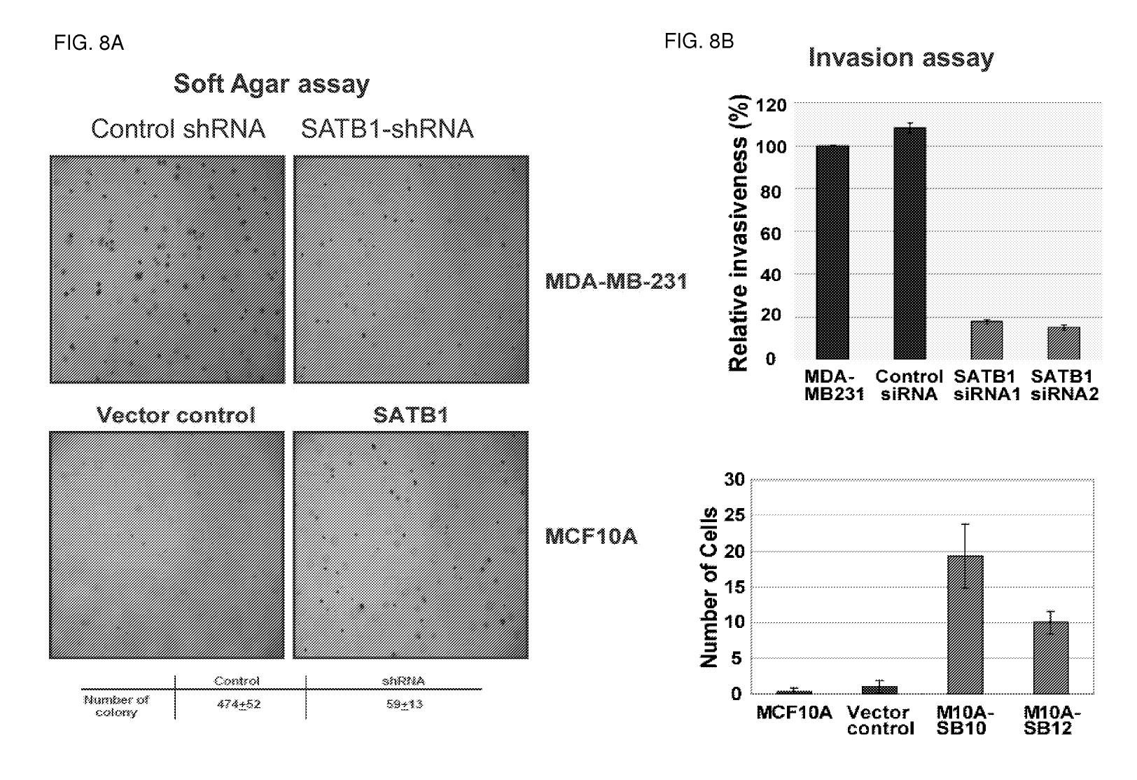

Reversal of invasive activity. Coincident with the morphology and formation of acini on Matrigel, depletion of SATB1 by shRNA reduced invasiveness of MDA-MB-231 host cells by 80-85%, as shown in the Boyden Chamber invasion assay in FIG. 8. When non-tumorigenic cells, MCF10A cells, are forced to express SATB1, these cells acquire the invasive activity.

Boyden Chamber Assay.

Boyden chamber chemo-migration assays⁵³ were performed using a 24-well chemotaxis chamber (BD biosciences, Inc.). Breast cancer cells were seeded in triplicate at 50,000 cells/well onto the upper chambers with a 8 μm polycarbonate filter membrane coated with diluted Matrigel (10-25%) (BD biosciences, Inc.), and incubated at 37° C. in humidified 5% CO₂ for 20 hrs. Conditioned media derived from NIH3T3 fibroblast cultures was used as a chemoattractant in the lower chambers. The migrated cells on underside of chambers were fixed in 10% (wt/vol) buffered-formalin and stained with crystal violet. After removal of cells remaining in the top chamber with a cotton swab, the numbers of cells that had migrated through the pores were assessed by light microscopy.

Loss of Anchorage-Independent Growth (Soft Agar Assay).

Cloned SATB1-depleted cells, as well as the SATB1-overexpressing cells, are subjected to a soft-agar cell growth assay, as previously described (See Kang, J. S., and Krauss, R. S. (1996) Mol Cell Biol 16, 3370-3380). Upon SATB1 depletion, MDA-MB-231 cells have lost anchorage-independent growth (characteristic of aggressive cells), while SATB1-forced expressed MCF-10A cells newly acquired anchorage-independent growth (FIG. 8)

### Example 4

### Global Change of Gene Expression by SATB1-shRNA in MDA-MB-231 Cells Either on Plastic Culture or on 3D Culture

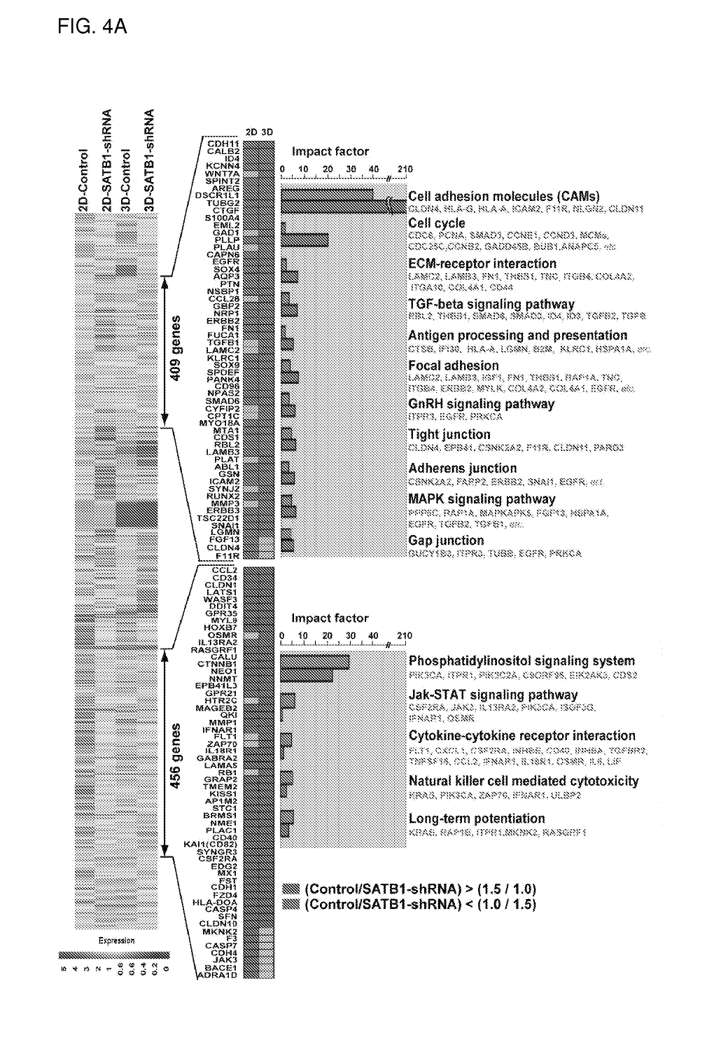

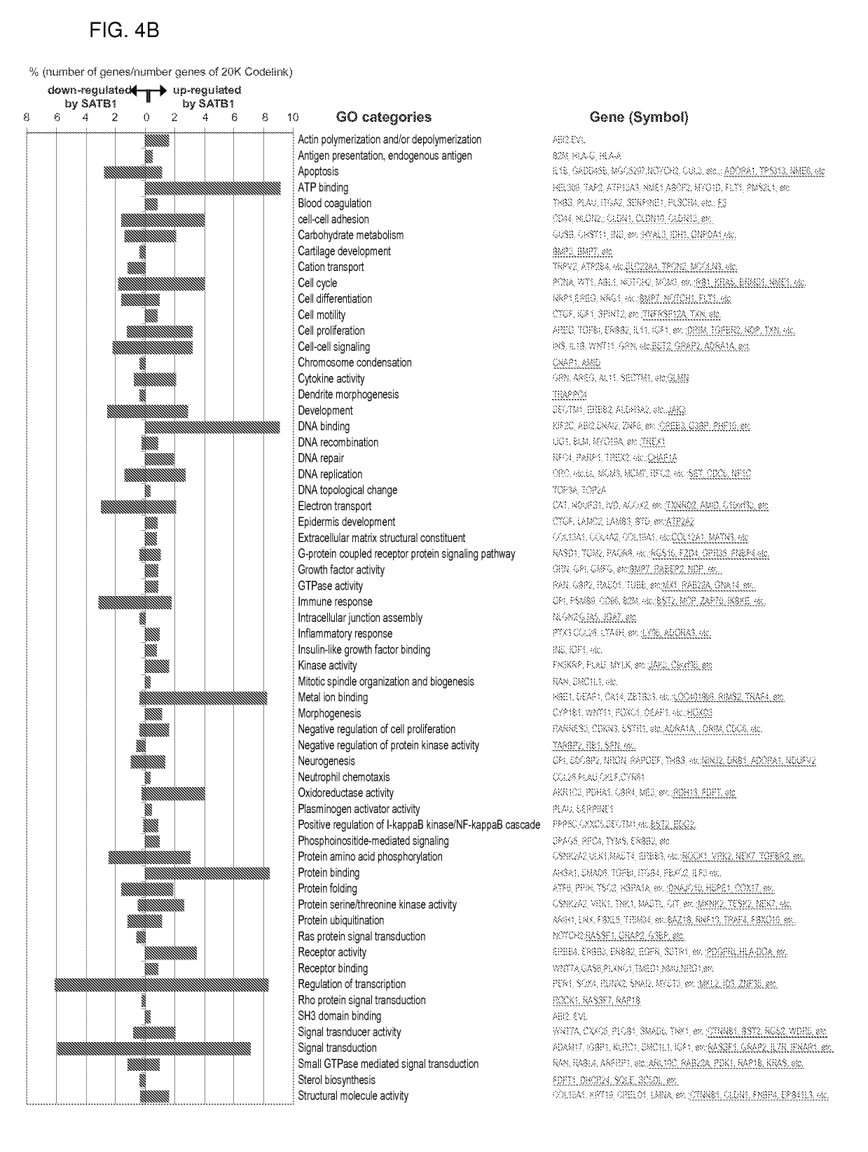

We performed gene expression profiling on MDA-MB-231 cells expressing either control shRNA or SATB1-shRNA1 grown in culture. Unsupervised clustering analysis of 2678 genes from two different microarray platforms (Affymetrix and Codelink) identified two groups of genes (Tree 1 and 2) that significantly changed expression levels (by>1.5-fold) following SATB1 depletion under both plastic and Matrigel culture conditions. Tree 1 included 409 downregulated genes, and Tree 2 contained 456 upregulated genes upon SATB1 depletion (FIG. 4a). We also categorized those affected genes based on the gene ontology (GO)(URL: <http//www.geneontology.org/index.shtml>) annotation. Functional profiling of these genes revealed that the greatest proportion of the genes were associated with cell adhesion, followed by phophatidylinositol signaling and cell cycle regulation. Individual SATB1-dependent genes and subgroups of different molecular pathways are shown in FIG. 4b.

Referring now to FIG. 4b, functional profiles using Gene Ontology terms for biological process and molecular function were constructed for SATB1-dependent up- and down-regulated genes (>2 fold) in either 2D or 3D cultures by Onto-Express (OE, at the URL http followed by: //vortex.cs.wayne.edu/ontoexpress) using the initial pool of 20,000 genes (Codelink from Amersham) as the reference set. The profiles for a total of 648 upregulated genes (red bar) and 519 down-regulated genes (green bar) were determined. The functional categories of biological process that were significantly represented by SATB1-dependent genes with the statistical significance of p<0.05 or represented by more than five genes were shown. The results show that SATB1-dependent genes are highly represented in most of the biological processes postulated to be associated with cancer including the positive control of cell cycle, cell proliferation, cell adhesion, signal transduction, cell-cell signaling and transcriptional regulation. The representative genes were listed as up-regulated (red) and down-regulated (green, underlined)..

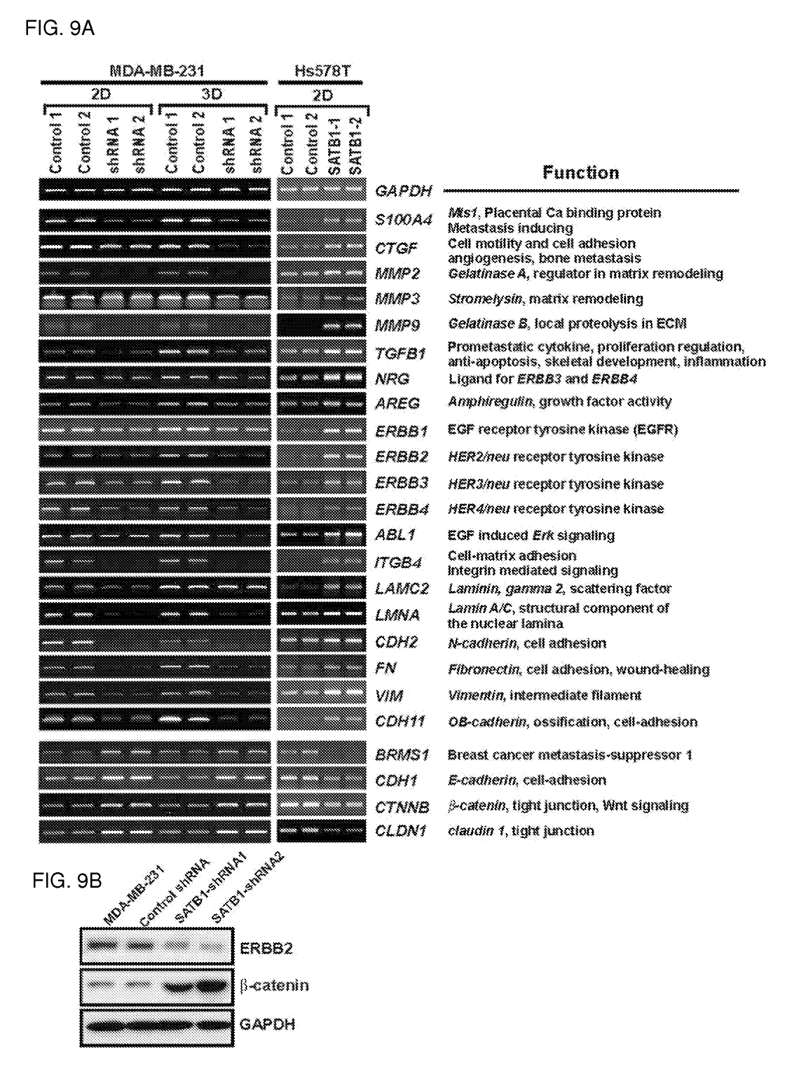

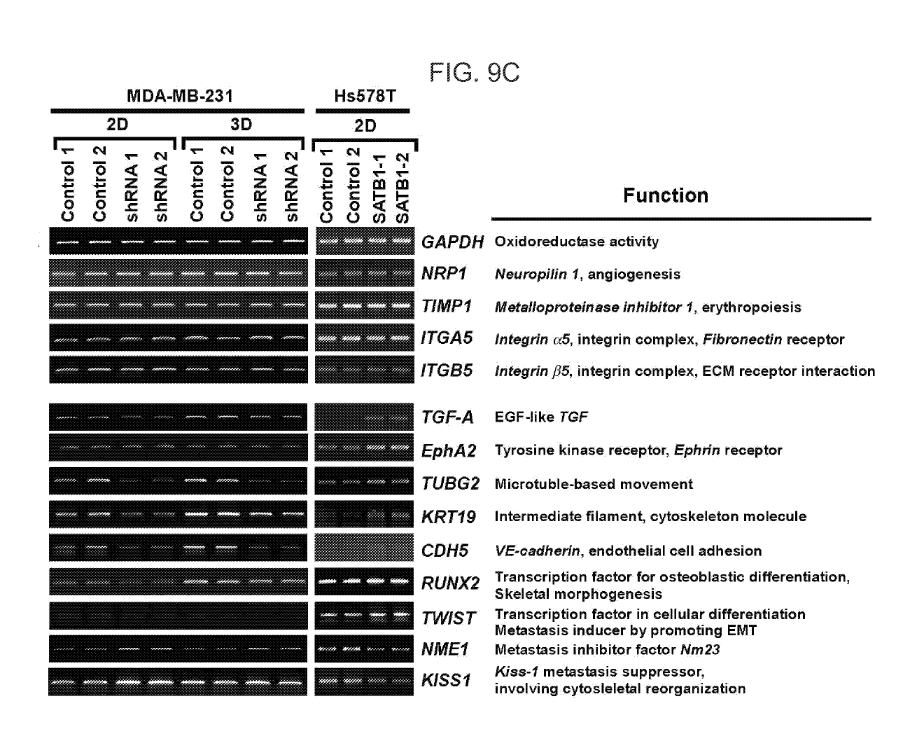

Based on both the data from our microarray analyses and from the published literature on genes associated with breast cancer progression, we selected over 40 genes to confirm their SATB1-dependent expression by RT-PCR (FIG. 9). In MDA-MB-231 cells, we found that SATB1 preferentially functions as a transcriptional enhancer, rather than as a repressor. Expression of many genes that are known to have important functions in promoting metastasis was found to be down-regulated upon SATB1 depletion. The SATB1-dependent genes associated with breast cancer progression include S100A4 (encodes Mts1 or metastasin) which has roles in metastasis and angiogensis; matrix metalloproteases (MMPs) 2, 3, and 9, which degrade extracellular matrix (ECM) and promote tumor invasion; tumor growth factor β1 (TGF-β1), which stimulates invasion; connective tissue growth factor (CTGF), which mediates angiogenesis and bone metastasis; and the tumor suppressor BRMS1 (FIG. 9). When SATB1 is expressed, the tumor suppressor BRMS1 is repressed, whereas all the other metastasis promoting genes in this group are upregulated.

Significantly, SATB1 expression also correlated with upregulation of genes involved in epidermal growth factor (EGF) signaling, such as EGF receptor subfamily members ERRB1, ERBB2 (also known as HER-2 or NEU), ERBB3, ERBB4, the ligands NRG and AREG, and the ABL1 oncogene, which has a role in EGF-induced-ERK signaling. ERBB2, the most oncogenic family member of ERBB protein, is an important regulator of breast cancer progression by coordinating the ERBB signaling network. Elevated expression of ERBB proteins are often found in human cancer and drugs that intercept signaling generated from ERBB2 are in routine clinical application. Many tumor cells exhibit increased invasiveness in response to TGF-β1 and increased levels of TGF-β1 has been reported in most tumor types. These results show that SATB1 promotes the expression of a set of genes that are known facilitators of metastasis while downregulating tumor suppressor genes. The dramatic shift in the gene expression pattern in cancer cells that express SATB1 can cause these cancer cells to acquire an invasive and aggressive phenotype.

Consistent with reversion of cell morphology for SATB1-depleted MDA-MB-231 cells, genes whose expression is up-regulated in invasive breast cancer and products contribute in cell structure are all down-regulated by SATB1 depletion (FIG. 9a). These genes include an ECM protein, fibronectin (FN); an intermediate filament protein, vimentin (VIM); cell-ECM interacting protein, β4 integrin (ITGB4). A nuclear structural protein, lamin A/C (LMNA), was similarly down-regulated by SATB1 depletion. Dysregulated expression in cadherin and catenins, which mediate cell-cell adhesion, has also been detected in breast cancer. OB-cadherin (CDH11), VE-cadherin (CDH5), and N-cadherin (CDH2) that are often up-regulated in invasive breast cancer were all repressed in SATB1 knock-down cells. Although SATB1 up-regulates the above described genes, for certain genes SATB1 acts as a repressor. These genes that are found de-repressed in SATB1 knock-down cells include CLDN1, a tight junction protein, which is known to be either lost or scattered in invasive tumors; β-catenin, a component of the cadherin-catenin complex and a critical member of the canonical Wnt pathway; E-cadherin, an adherens junction protein and tumor suppressor. Loss of E-cadherin is a hallmark for epithelial to mesenschymal transition (EMT)—a process whereby epithelial cells layers lose polarity and cell-cell contacts and undergo a dramatic remodeling of the cytoskeleton, which is believed to contribute to the dissemination of carcinoma cells from epithelial tumors. SATB1 depletion from MDA-MB-231 cells resulting in upregulation of E-cadherin and restoration of acinar-like morphology strongly suggest that the EMT process was reversed

### Example 5

### Identification of Genes Directly Regulated by SATB1

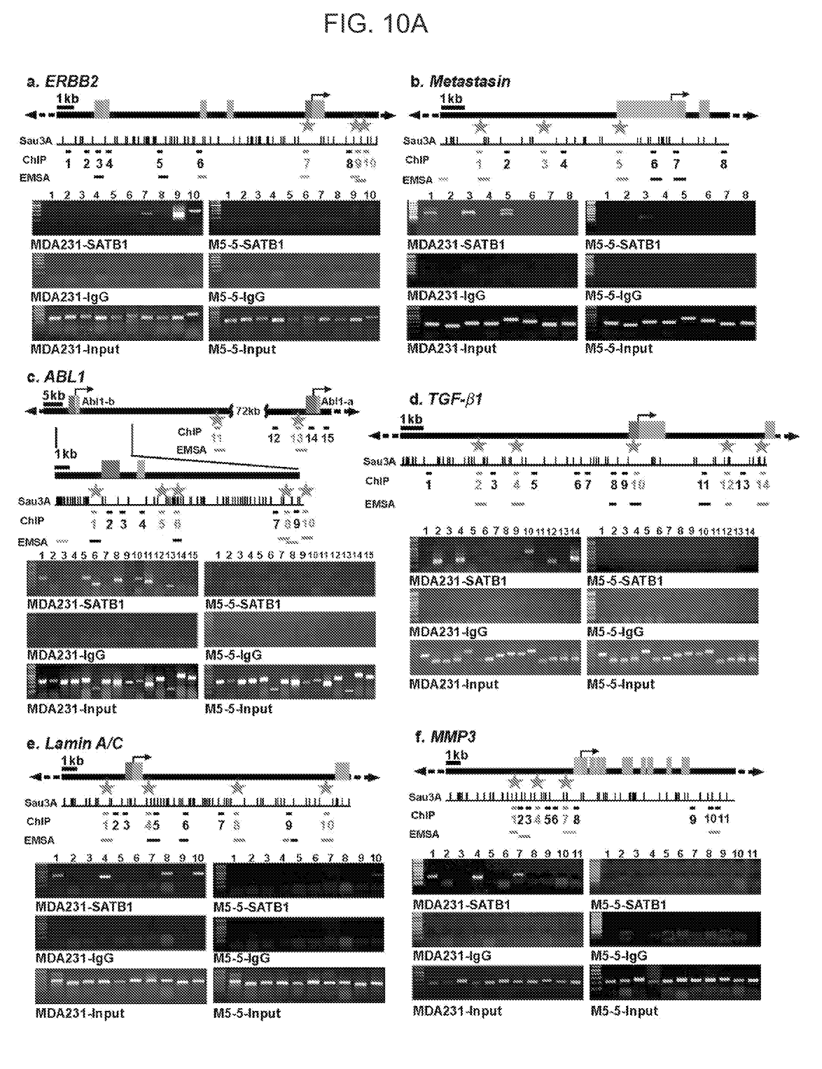

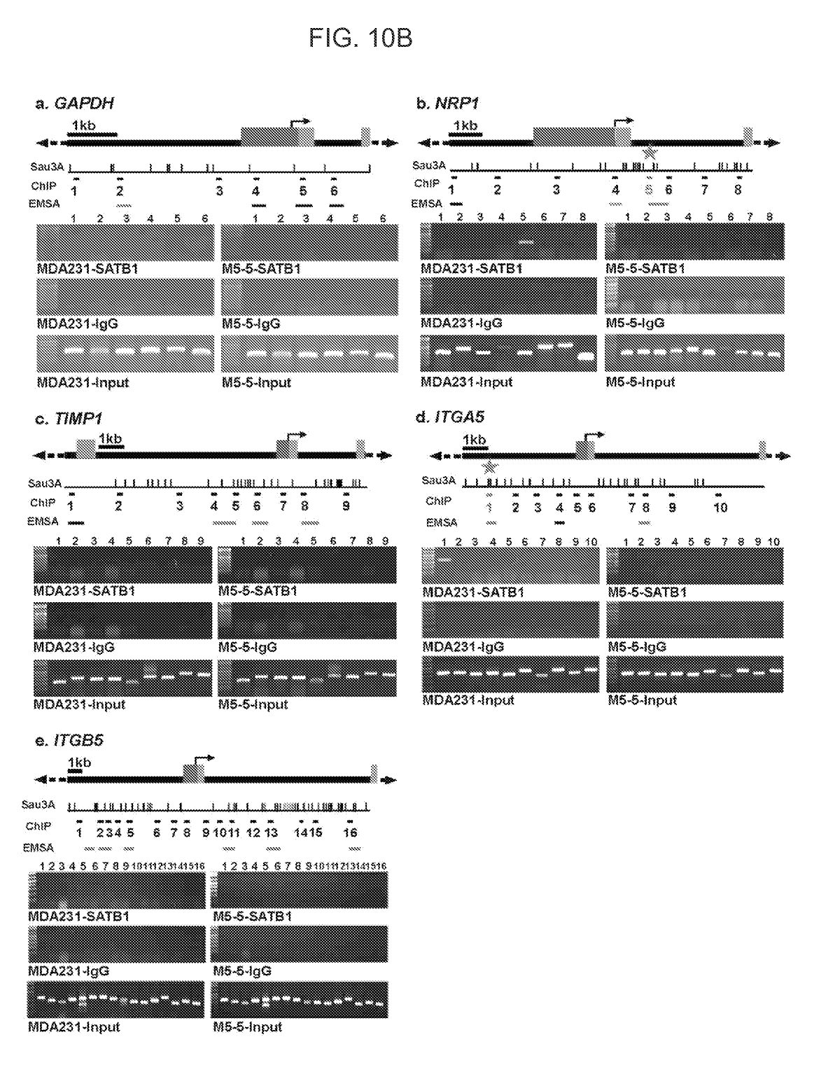

To identify genes directly regulated by SATB1, we determined the in vivo binding status of SATB1 within genomic loci of 6 genes whose expression was correlated with SATB1 levels in cells. These genes are ERBB2, Metastasin, ABL1, TGF-β1, LaminA/C, and MMP3, representing candidate genes directly regulated by SATB1 (FIG. 10a). Five genes whose expression was observed to be independent of SATB1 levels were selected as non-SATB1 target controls (GAPDH, NRP1, TIMP1, ITGA5, and ITBG5) (FIG. 10b). For each of these selected genes, we analyzed a ˜15 kb region upstream and downstream of the gene's first exon for SATB1 binding in vivo, looking for all potential SATB1 target sequences (BURs), promoter sequences (if known), regions containing CpG islands, and other control sequences that would not be predicted to bind SATB1 based on DNA sequence. Potential BURs could be identified by the genomic sequences characterized by the ATC sequence context. SATB1 binding to each of these candidate sites was confirmed by electrophoresis mobility shift assay (EMSA). To assess SATB1 binding to these loci in vivo, we employed the urea-ChIP method described in Kohwi-Shigematsu, T., deBelle, I., Dickinson, L. A., Galande, S. & Kohwi, Y. Identification of base-unpairing region-binding proteins and characterization of their in vivo binding sequences. Methods Cell Biol 53, 323-54 (1998) and below, in which chromatin was crosslinked, purified from the cell lines by urea-gradient centrifugation, Sau3A digested, and SATB1-containing chromatin fragments were immunoprecipitated using an anti-SATB1 antibody.

A urea-chromatin immunoprecipitation (ChIP) on promoter chip approach in combination with cDNA microarray experiments was used to identify a large number of genes that are directly regulated by SATB1 in breast cancer cells. Chromatin immunoprecipitation (IP) and a promoter array which is now available from the Microarray Centre, Universiy Health Network, Canada were combined. This microarray contains 12,192 CpG-island sequences enriched in promoter sequences. The pool of DNA sequences from chromatin IP against anti-SATB1 antibody in MDA-MB-231 cells will be used as hybridization probes for the human promoter microarray. The rationale for using this approach to identify the direct target gene of SATB1 is the following: using our modified ChIP assay, it was previously found that SATB1 anchors specialized DNA sequences (ATC sequence context) in the target gene loci, that are found often in the upstream regions (up to 60 kb of the promoters) and within introns. However, it was also found that at certain higher cycles of ligation-mediated PCR (LM-PCR) of ChIP fragments. Thus, detection of the promoter regions of the target genes, but not target genes, to be bound by SATB1 can be carried out. This less strong, but definitive binding of SATB1 to the target gene promoter is probably dynamic and accomplished by local chromatin looping by SATB1 tethering to the promoter mediated by transcription factors.

The urea-ChIP method, which was devised by our lab, involves extensive purification of cross-linked chromatin from non-cross linked proteins and RNAs by urea gradient centrifugation. Urea-ChIP experiments were performed as previously described with modification as described in Yasui, D., Miyano, M., Cai, S., Varga-Weisz, P. & Kohwi-Shigematsu, T. SATB1 targets chromatin remodelling to regulate genes over long distances. Nature 419, 641-5 (2002), and de Belle, I., Cai, S. & Kohwi-Shigematsu, T. The genomic sequences bound to special AT-rich sequence-binding protein 1 (SATB1) in vivo in Jurkat T cells are tightly associated with the nuclear matrix at the bases of the chromatin loops. J Cell Biol 141, 335-48. (1998).

Cross-linked chromatin by formaldehyde was isolated from MDA-MB-231 and SATB1-shRNA MDA cells (M5-5), and then purified further using urea-gradient ultracentrifugation. After 60 U of Sau3A1 digestion, we performed immunoprecipitation of 30 μg of cross-liked chromatin against anti-SATB1 antibody (BD Biosciences) and purified mouse IgG₁ (Sigma) as a control. We reversed the ChIP samples with 100 μg/ml RNase A and 250 μg/ml proteinase K treatment, followed by incubation at 65° C. for 6 hrs. We carried out PCR reaction using reverse cross-linked chromatin with AmpliTag-Gold DNA Polymerase (Applied Biosystems) in following cycling condition; 95° C. for 10 min and 35-40 cycles of denaturation at 95° C. for 10 s, annealing at 56° C. for 20 s, and extension at 72° C. for 30 s using GeneAmp PCR system 9700 (PerkinElmer Inc.). We designed the primer sequences mainly focused on the promoter regions of each gene (covering ˜15 kb) using Vector NTI software (Invitrogen. Gel mobility shift assay (EMSA) were performed to determine the in vitro binding ability between SATB1 and SATB1 binding sequences in vivo, which we detected in urea-ChIP assay. The results obtained from urea-ChIP on promoter chip will be confirmed by examining the expression of these genes in the cDNA microarray analysis data and making sure that it is altered in SATB1-depleted MDA-MB-231 cells in comparison to wild-type MDA-MB-231 cells. The cDNA microarray data was readily available, and therefore, this ChIP on promoter chip provides for the first time evidence for the function of SATB1 as a global gene regulator in aggressive breast cancer cells and its specific set of target genes in these cells.

By comparing the overall in vivo SATB1-binding statuses for the candidate SATB1-target gene loci versus the non-target control gene loci, a striking contrast was found in SATB1 binding patterns between the two groups (FIG. 10a, b). In all six candidate target gene loci, virtually all SATB1-binding sequences, predicted based on the ATC sequence context and confirmed by EMSA, were bound to SATB1 in vivo (FIG. 10a). A similar binding pattern was detected for the β-catenin locus, whose expression was downregulated when SATB1 was overexpressed (data not shown). On the other hand, in non-target control gene loci, even though all of them contained many sequences intrinsically capable of binding to SATB1 in vitro, SATB1 was rarely found to be associated with them in vivo (FIG. 10b). The contrast in the in vivo binding frequencies of SATB1 between the two sets of genes indicates that there are at least two clearly different gene subgroups, that can be distinguished by the SATB1-binding status, regardless of whether SATB1 represses or activates expression of the genes. These results, taken together with SATB1-dependent expression, indicates that SATB1 directly regulates expression of ERBB2, Metastasin, ABL1, TGF-β1, LaminA/C, MMP3 and β-catenin. We also determined the in vivo binding status of SATB1 within genomic loci of SATB1-dependent genes BRMS1, CLDN1, and CTNNB1, which are downregulated by SATB1. The results indicated that SATB1 also directly regulates expression of these gene.

For genes directly regulated by SATB1, SATB1 binding does not occur exclusively at the sequences that have the capacity to bind SATB1. Some sequences near promoters or CpG islands that totally lack SATB1 binding potential based on EMSA can be bound in vivo. Such binding sites are found near promoters of ABL1 (sites 1 and 6), TGF-β1 (site 10), Lamin A/C (site 4) (FIG. 10a). The remaining SATB1 target genes tested here already have SATB1-binding sequences near promoters, and SATB1 binds to these sites in vivo. SATB1 binding in vivo to promoters or nearby sequences is another hallmark for direct target genes. This is because SATB1 binding indirectly to promoter/regulatory sequences has been found within other known direct target genes of SATB1, such as Il2Rα and 114, 115, 1113. Such indirect binding by SATB1 presumably reflects the SATB1-mediated formation of a large genomic DNA/protein complex placing multiple genomic sites into close spatial proximity. Many other metastasis or cancer-associated genes whose expression is SATB1 dependent are likely to exhibit similar pattern for in vivo association with SATB1.

### Example 6

### SATB1 is Required for Metastasis of MDA-MB-231 Cells to Lung and is Necessary for Tumor Growth

To determine whether metastasizing activity of breast cancer cells depends on SATB1 expression in vivo using a mouse model. We address this question by determining whether MDA-MB-231 cells lose their metastasizing activity upon depletion of SATB1 expression by shRNA. We further addressed whether overexpression of SATB1 in less aggressive Hs558T cells increases metastatic activity in vivo.

Because SATB1 knock-down reduces breast cancer cell invasion and colony formation in soft agar and restores normal cell morphology in vitro, we evaluated its in vivo effects on metastasis using experimental metastasis assay using nude mice.

The Removal of SATB1 from MDA-MB-231 (Aggressive) Cells Diminished Metastatic Activity:

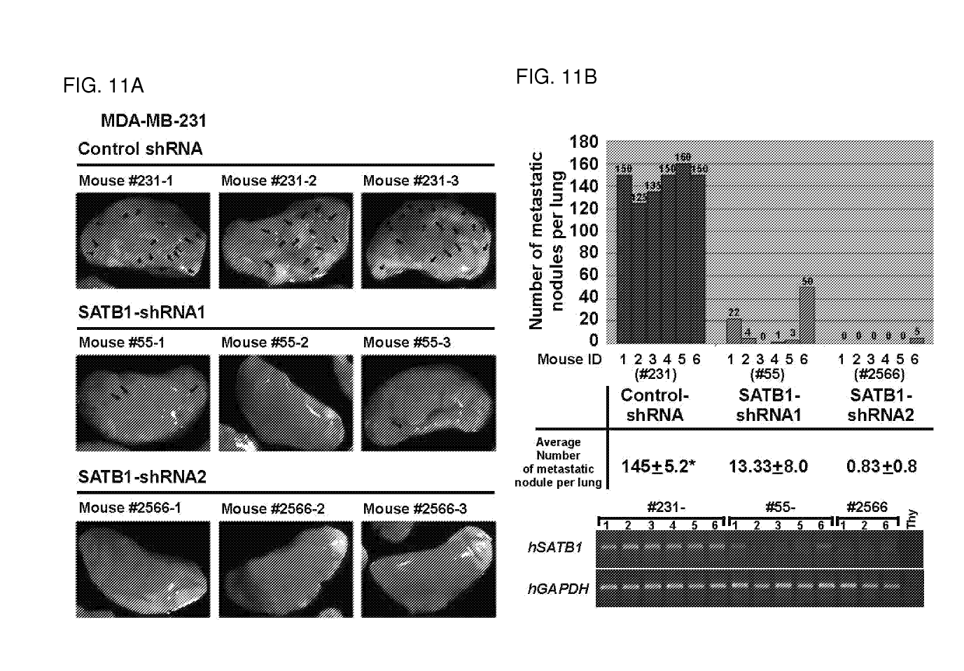

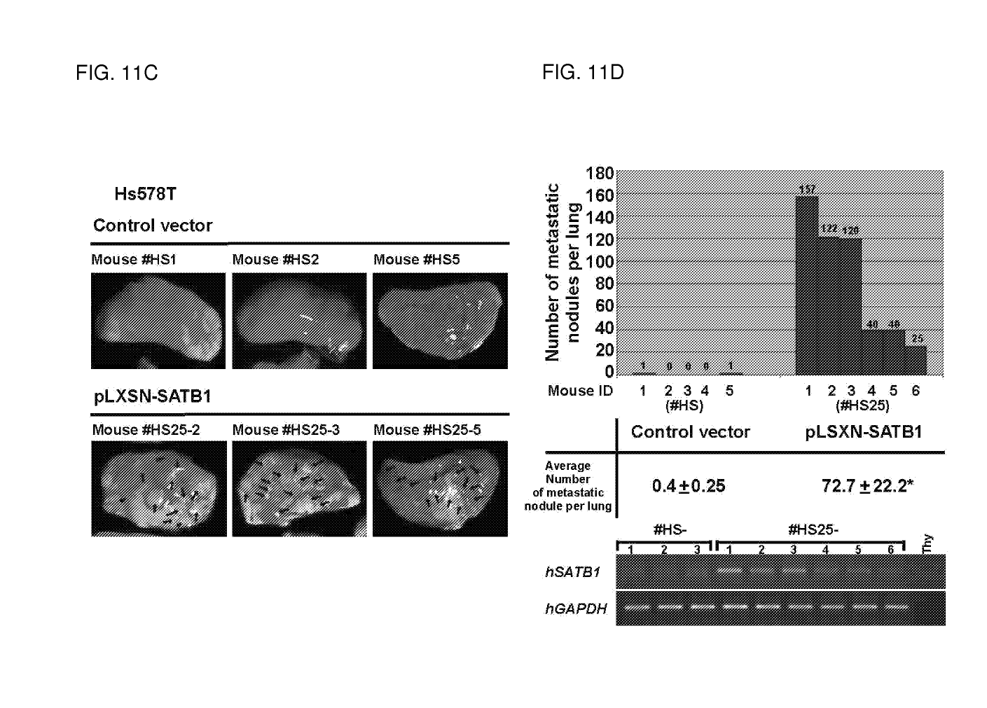

SATB1-shRNA1 MDA, SATB1-shRNA2 MDA, and control cells (1×10⁶ cells) were injected intravenously into the lateral tail vein of 6-week-old athymic mice to evaluate metastasis of these cancer cell lines to lung. In mice, the metastasis of orthotopically grown tumors derived from human MDA-MB-231 cells is a relatively rare event. Therefore, by directly introducing cells into the circulation, we examined the requirement of SATB1 in cancer cell survival in circulation and extravasation to and growth in the lung. By nine weeks after tumor-cell injection, the lungs of mice injected with the control cells had formed numerous nodules, ranging in number from 125 to 160 per lung in all six mice analyzed (FIG. 11a,b). In contrast, the number of lung metastases was greatly reduced in mice injected with the SATB1 knock-down cells, SATB1-shRNA1 MDA cells, ranging from 0 to only 50 per lung among six mice. The lung metastases derived from the SATB1-depleted tumor cells were also much smaller in size than those derived from the control cells. Lung metastases from the second knock-down cell line, SATB1-shRNA2 MDA cells, were not observed in five out of six mice, and one mouse injected with these cells developed only five nodules/lung (FIG. 11b). Thus, our data from experimental metastasis analyses indicate that SATB1 is necessary for the aggressive, highly metastatic phenotype of MDA-MB-231 cells and suggest that the levels of SATB1 also play an important role in the metastatic activity of cancer cells.

As shown in FIG. 11a, RNAi-mediated depletion of SATB1 inhibited the ability of MDA-MB-231 cells to metastasize to lungs of nude mice. 1×10⁶ cells MDA-MB-231 cells expressing control-shRNA, SATB1-shRNA1 or SATB1-shRNA2 MDA cells were injected into the tail veins of each mouse, and lungs were examined for metastatic nodules (arrows) 9 weeks later. Representative photos are shown. In FIG. 11b, total numbers of metastatic lung nodules from individual mice were counted under a dissection microscope. For lungs of representative mice indicated, human SATB1 expression levels in human breast cancer cells colonized in lungs were analyzed by RT-PCR using human GAPDH as a loading control, with the use of human SATB1 and GAPDH specific oligomers. The specificity of these oligomers for human genes is shown by the absence of RT-PCR signals for mouse thymcoytes (Thy). Thus, it is demonstrated that in vivo expression of SATB1-shRNA inhibits SATB1 expression and thereby inhibits metastasis.

We next tested whether SATB1 depletion from MDA-MB-231 cells also inhibits tumor growth. We injected control and SATB1-shRNA1 cells into the fourth mammary fat pads of athymic nude mice and monitored tumor growth. In contrast to both parental MDA-MB-231 and control-shRNA cells, which formed large tumors within 39 days (6/6 mice), all 6 mice injected with an SATB1-shRNA1 clone or a pool of SATB1-shRNA1 cells resulted in either no tumors or greatly reduced tumor growth, respectively. These results indicate that SATB1 expression in MDA-MB-231 cells is necessary for the tumor growth of these cells in mammary fat pads of mice.

SATB1 Overexpression in Hs578T Cells Increases Metastasis.

We also tested whether SATB1 overexpression could promote metastasis in another breast cancer cell line, Hs578T, in which SATB1 is expressed at a lower level than in MDA-MB-231 cells. When we forced expression of SATB1 at higher levels in Hs578T cells, their invasive activity in vitro was greatly increased (FIG. 11c,d). When control Hs578T cells (2×10⁶ cells/mouse) transfected with vector alone were injected into 6 mice (HS), only two mice developed metastatic nodules in the lung and in both cases there was only one nodule per mouse, consistent with the less aggressive nature of the Hs578T cells than the MDA-MB-231 cells (FIG. 11c, d). In contrast, Hs578T cells transfected with pLXSN-SATB1 and over-expressing SATB1 formed a greatly increased number of lung metastases in all mice (HS25), ranging from 25 to 157 metastatic nodules per lung. Of 6 mice injected with the SATB1-overexpressing cells, 3 mice developed over 120 metastatic nodules per lung. This number was equivalent to the number of metastases that formed in the lungs of mice injected with control MDA-MB-231 cells. Therefore, the results strongly suggest that SATB1 is not only required for, but induces breast cancer cell metastasis to lung (FIG. 11c, d).

Overexpression of SATB1 promotes the ability of Hs578T cells to metastasize to lungs. 2×10⁶ Hs578T cells transfected with vector alone (control) or with an SATB1 expression construct (pLXSN-SATB1) were injected into each mouse, via the tail vein, and lungs were examined 9 weeks later. Representative photos from three independent mice are shown in FIG. 11c.

SATB1 Expression in Non-Metastatic Cancer Cells Induces Invasive Activity.

We examined whether ectopic expression of SATB1 is sufficient to induce invasive activity in non-metastatic cancer cells. Control SKBR3 cells (a non-metastatic cell line, transfected with control vector) injected into the mammary glands of mice did not form tumors in mice after 7 weeks. In contrast, all 6 mice similarly injected with SKBR3 cells ectopically expressing SATB1 (pLXSN-SATB1) grew large undifferentiated, highly vascularized tumors. To examine intravasation, cells isolated from blood and lung tissue from mice injected with SKBR3 cells (control or pLXSN-SATB1) were cultured for 4 weeks in the presence of G418, to select for transfected cells. In 5 out of the 6 mice injected with SKBR3 cells, 2 to 23 colonies formed from each blood sample, and in all these mice 2 to 11 colonies formed from each lung samples. No colonies grew from samples from mice injected with control cells. These data show that expression of SATB1 is sufficient to induce SKBR3 cells to form large tumors in mammary fat pads, to acquire the ability to invade blood vessels, and to survive in the circulation.

By 7 weeks post-injection, we did not observe macroscopically visible metastases in the lungs of mice injected with SATB1-expressing cells in the mammary fat pads; longer monitoring times would be needed to observe such secondary tumors. However, we had to sacrifice mice bearing large tumors after 7 weeks. Therefore, we intravenously injected SKBR3 cells (control vector or pLXSN-SATB1) into mice and found that at 10 weeks post-injection, SATB1 expressing SKBR3, but not control SKBR3 cells, formed many large metastatic nodules, indicating extravasation and tumor growth in lung.

Tumorigenic Activity of SATB1.

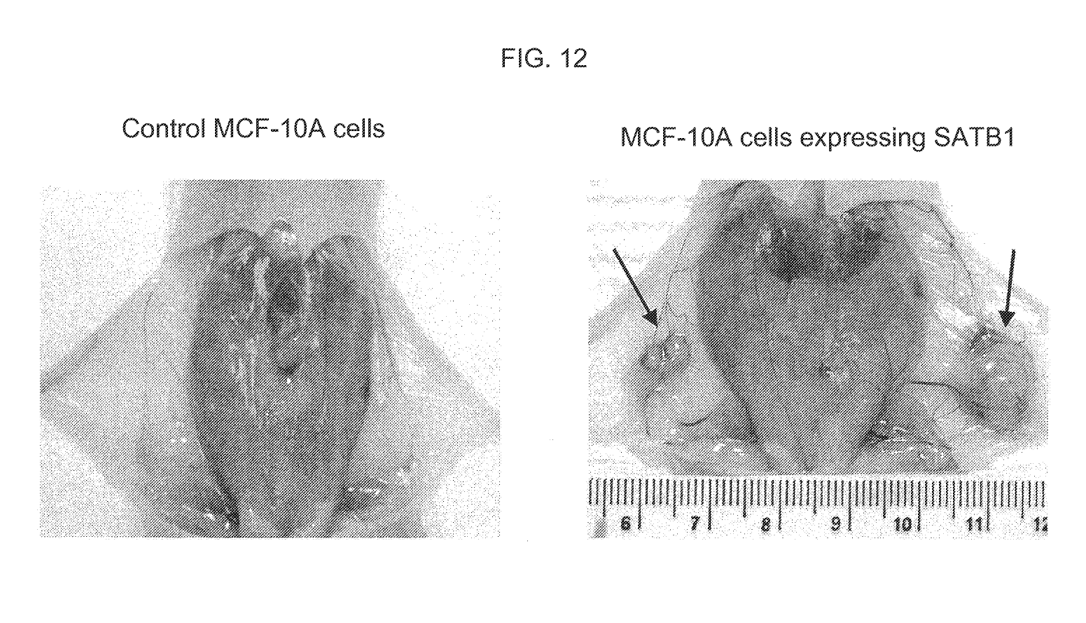

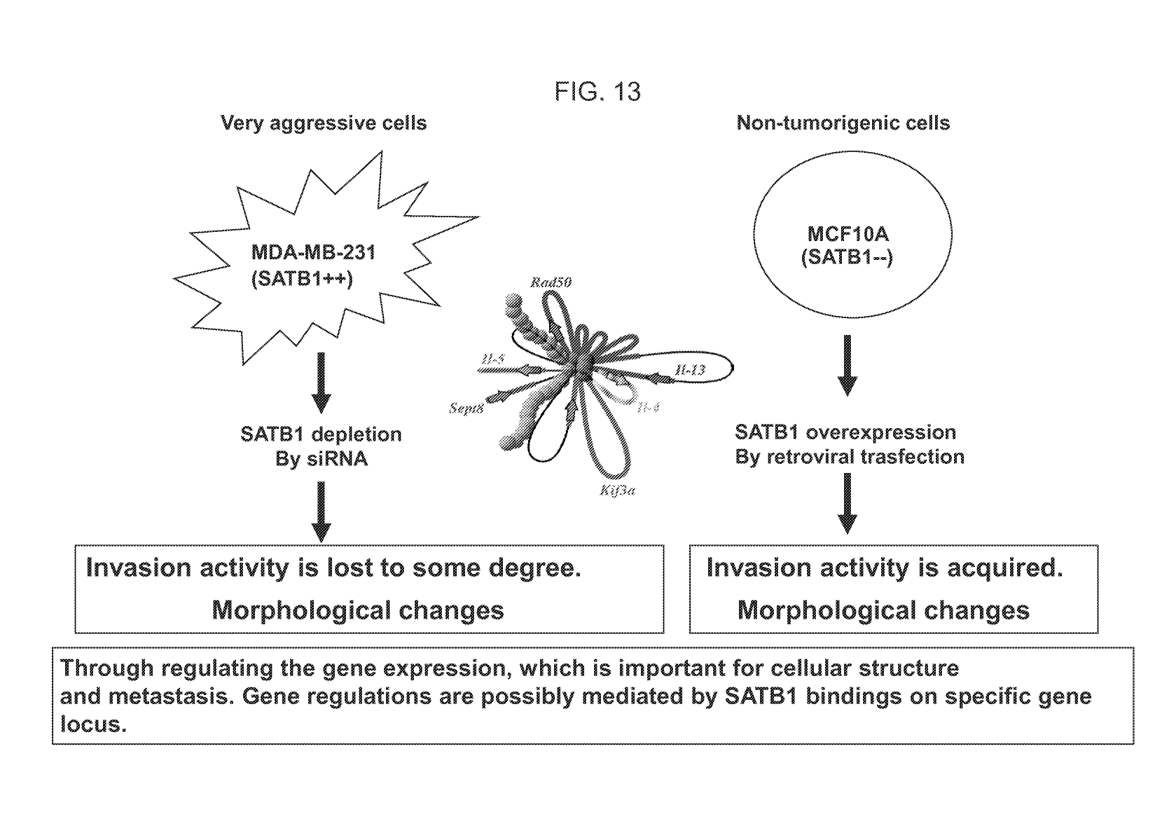

We evaluated the activity of tumor formation by ectopic expression of SATB1 in non-tumorigenic cells. Forced expression of SATB1 in non-tumorigenic MCF-10A cells generated breast tumors in nude mice. We injected MCF-10A cells stably transfected with the SATB1 expression construct into the fat pads of nude mice. After 9 weeks, we sacrificed the mice. In all of the six mice transfected with MCF-10A expressing SATB1 had tumors, while none of the mice transfected with MCF-10A stably transfected with control vector construct showed tumors (FIG. 12). Thus, it is shown that in vivo expression of the SATB1 promotes tumorigenicity.

### Example 7

### An Assay System for Screening SATB1 Inhibitors, and Testing the Biological Effect of Selected Compounds

In this example, we describe the development of an assay system for screening small chemical molecules available at MLSCN, to identify chemicals which successfully block SATB1 activity in aggressive breast cancer cells, and 2) test the biological effect of selected compounds on the invasive property of aggressive breast cancer cells.

In an aggressive breast cancer cell line, MDA-MB-231, we determined that SATB1 directly regulates oncogenes such as ERBB2 and ABL1, and tumor suppressor genes, such as KISS1, CDH1, BRMS1, and NME1 (data shown in FIG. 9C and described above). In fact, depletion of SATB1 by RNAi resulted in a major reduction in the invasive property of the MDA-MB-231 cells and change in their cell morphology on Matrigel. Thus, it is possible to develop an assay to screen chemicals which will target SATB1 activity in aggressive breast cancer cells. The Molecular Libraries Roadmap of NIH will offer researchers access to small organic molecules that can be used as chemical probes, to study the functions of genes and to facilitate the development of new drugs. The Molecular Libraries Screening Center Network (MLSCN) will accept assays for high-throughput screening (HTS) to screen ˜500,000 chemically diverse small molecules.

Thus, it is highly likely that a sensitive and accurate in vitro assay system can be developed to identify chemicals that interfere with the SATB1/BUR interaction. We will place well-characterized BUR sequences derived from the immunoglobulin heavy chain (IgH) enhancer BUR in the expression vector containing a gene encoding the green fluorescent protein (GFP). In the absence of SATB1, the BURs greatly augment the reporter gene expression in stable transformants. Because SATB1 is known to bind BURs and inhibit the reporter transcription, the GFP protein should be repressed and not visible when SATB1 is present. However, in the presence of a chemical which inhibits SATB1 repressor activity, the GFP protein should become expressed. Therefore, the presence of such a chemical can be detected by the appearance of green fluorescence. Such an assay system will provide a quick screening of a huge number of chemicals. The positive chemicals will be tested for their effects on the invasive activity in vitro.

Methods:

We will use an enhanced GFP (EGFP) reporter construct in which IgH 3′ BUR sequences are inserted at 5′ and 3′ of the reporter EGFP gene. We will also make, as a control, a mutation construct containing the mutated version of the BUR which has lost the unwinding propensity. Either wild-type BUR or mutated BUR expression cassettes will be stably transfected alone or co-transfected with a SATB1-expression construct into various cell lines which do not normally express SATB1. We will establish a stably transfected cell system by these two criteria: 1) EGFP surrounded by wild-type BURs is strongly expressed, but it is not expressed when surrounded by the mutated BURs, 2) in the presence of the co-transfected SATB1 expression construct, EGFP surrounded by wild-type BURs is completely repressed, but not with mutated BURs. If SATB1 expression causes cell toxicity in a long-term culture, we will employ an inducible expression system. Once stably transfected cell clones are established satisfying the above two criteria, we will use a cell clone containing EGFP surrounded by wild-type BURs co-transfected with the SATB1 expression vector, to screen chemicals which block the repressor activity of SATB1, as indicated by highly fluorescent cells. For those chemicals which are potential inhibitors SATB1, we will study their effects on the aggressive phenotype using the Boyden Chamber invasion assay.

### Example 8

### Comparison with the Prognosis Signature Genes with SATB1-Regulating Genes in Microarray

Gene expression profiling of human breast carcinomas have identified characteristic gene expression patterns often associated with poor prognoses. See Ramaswamy, S., Ross, K. N., Lander, E. S. & Golub, T. R. A molecular signature of metastasis in primary solid tumors. Nat Genet 33, 49-54 (2003); van de Vijver, M. J. et al. A gene-expression signature as a predictor of survival in breast cancer. N Engl J Med 347, 1999-2009 (2002); and van't Veer, L. J. et al. Gene expression profiling predicts clinical outcome of breast cancer. Nature 415, 530-6 (2002). The presence of such poor-prognosis gene signatures, detectable even in primary tumors, challenges the long-held view that metastatic cells are rare and evolve during late stages of tumor progression via a series of genetic changes. Indeed there is little mechanistic evidence for how poor prognosis-associated gene expression profiles arise. Such expression patterns may arise fortuitously in some cells in primary tumors, or a functional mediator may be newly expressed that specifically directs changes in the expression pattern of the primary tumor cells, resulting in a metastatic phenotype.

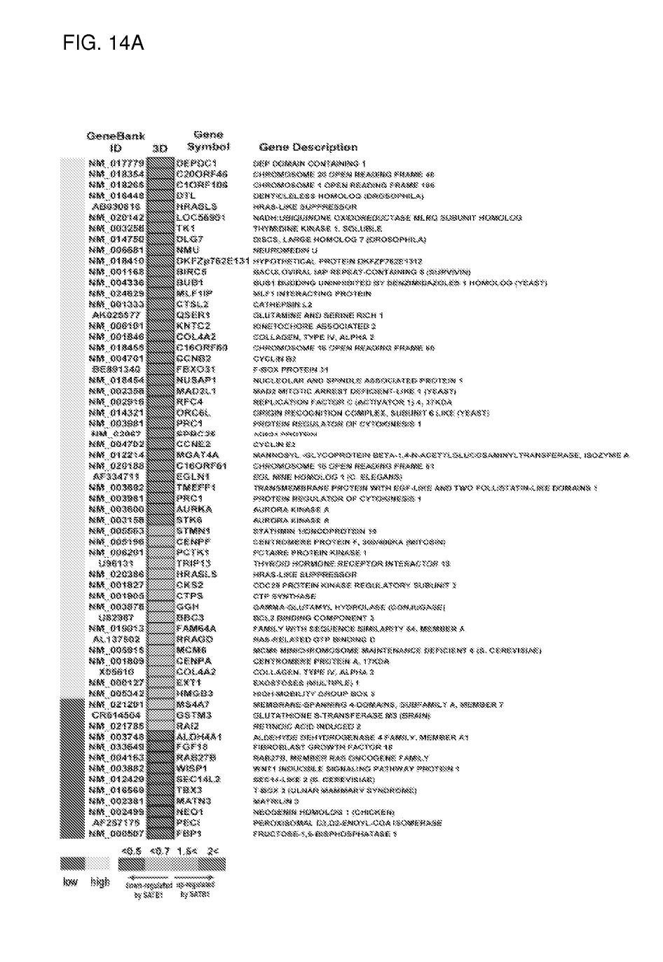

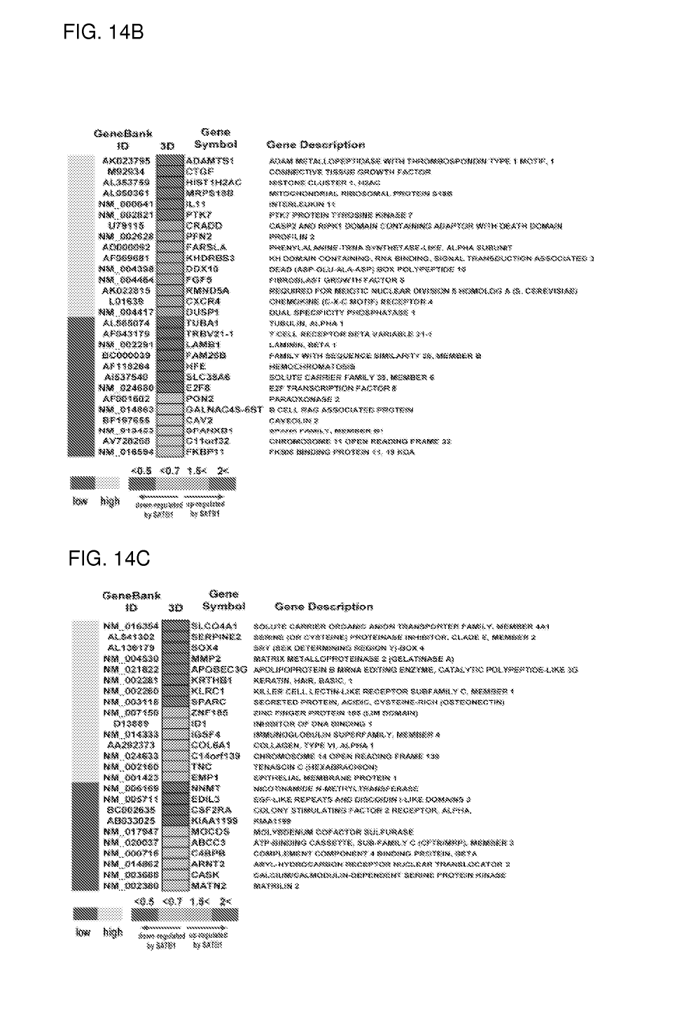

Among the 231 Rossetta poor-prognosis associated genes (van't Veer, L. J. et al., Nature 415, 530-6 (2002)), 174 were compared by our microarray to the SATB1-dependent gene set (shown in Trees 1 and 2, FIG. 5a). Sixty-three of these genes (36%) whose expression was up- or down-regulated in breast tumors with poor prognoses were correspondingly altered by SATB1 expression (P=0.02) (FIG. 14a). Genes known to promote either bone or lung metastasis were also enriched among the SATB1-dependent genes in MDA-MB-231 cells (P=0.0002, P=0.021, respectively (FIG. 14b,c).

As shown in the previous Examples, the statistical data for the association of strong SATB1 expression and aggressive, poorly differentiated breast cancer shows P<0.001 using a total of 38 tissues samples (10 normal, 5 moderately differentiated, and 23 poorly differentiated). To further confirm the diagnostic values of SATB1 for clinical use, as well as to examine its prognostic significance, we will use The National Cancer Institute (NCI) Cooperative Breast Cancer Tissue Resource (CBCTR) which is funded by the NCI to supply researchers with primary breast cancer tissues with associated clinical data. The CBCTR can provide formalin-fixed, paraffin-embedded primary breast cancer specimens with their associated pathologic and clinical outcome information (i.e. Follow-up time up to 10 years, tumor size, nodal status, histologic type, vital status, treatment received, recurrence information, stage of disease). This valuable finite collection is intended to facilitate large research studies that require archival tissue with clinical and outcome data.

Upon confirmation using the CBCTR specimens, SATB1 can be for prognostics and diagnostic use. For example, a biopsy of a primary tissue from a lymph node negative patient is also immuno-stained with SATB1 antibodies to detect SATB1. If SATB1 is positively found, then the patient should be considered for more aggressive anti-cancer treatment, such as radiation and/or chemotherapy.

### Example 9

### Down Regulation of SATB1 in a Subject

In Vivo Studies in Human Subjects.

SATB1 shRNA Preparation and Treatment: Suspensions of the shRNAs of Example 4 can be prepared by combining the oligonucleotides and a buffer or detergent to prepare suspensions in a therapeutic concentration range. The siRNA is synthesized, weighed and can be dissolved in low salt buffer through mixing and sonication. Solubilizing and delivery agents can be added to the solution. Dilutions can be made from a stock solution and the final excipient, such as 0.9% NaCl at 37° C., is added to each dose formulation just prior to dosing. The final ratio of liquid components (e.g., buffer, siRNA, and saline) can be, for example, 5:5:90, respectively. Subjects having been diagnosed with aggressive cancers where SATB1 is detected as expressed ectopically in malignant cells, can be given a therapeutically effective amount of the solution interstitially or intratumorally. A sample dosage may be 0.1 to 0.5 ml, one to five times/week, using a syringe and a needle.

After sufficient period of siRNA administration, a noticeable decrease in the tumor cell growth and cell division should be observed. Administration of the shRNA should cause depletion of SATB1 in the tumor cells, thereby prohibiting the metastasis and growth characteristic of aggressive tumor cells.

The present examples, methods, procedures, specific compounds and molecules are meant to exemplify and illustrate the invention and should in no way be seen as limiting the scope of the invention. Any patents, publications, publicly available sequences mentioned in this specification are indicative of levels of those skilled in the art to which the invention pertains and are hereby incorporated by reference to the same extent as if each was specifically and individually incorporated by reference.

1. A prognostic method for identifying a patient that has a cancer that is a candidate for aggressive treatment, said method comprising:
providing a primary tumor tissue biopsy from a patient;
detecting SATB1 expression, whereby said detection of SATB1 expression is a predictor or marker of that the cancer is aggressive.

2. The method of claim 1 wherein the cancer is breast cancer.

3. The method of claim 1 wherein the assay is an immunochemical assay to detect SATB1 protein levels.

4. The method of claim 1 wherein the assay is an RT-PCR assay to detect SATB1 transcription levels.

5. A method of inhibiting SATB1 expression in a tumor cell, the method comprising administering a SATB1 inhibitor.

6. The method of claim 5 wherein the inhibitor is an antisense oligonucleotide.

7. The method of claim 5 wherein the inhibitor is a siRNA olignonucleotide.

8. The method of claim 7 wherein the siRNA oligonucleotide is selected from the group consisting of: SEQ ID NO: 4, SEQ ID NO: 5, SEQ ID NO: 6, and SEQ ID NO: 7.

9. The method of claim 5 wherein the inhibitor is a small molecule that interferes with SATB1 function.

10. The method of claim 5 wherein the inhibitor is a viral vector producing a nucleic acid sequence that inhibits SATB1.

11. The method of claim 5 wherein the inhibitor is an aptamer.

12. The method of claim 5 wherein the inhibitor is an antibody.

13. The method of claim 5 wherein the inhibitor is a shRNA oligonucleotide.

14. The method of claim 13, wherein the shRNA oligonucleotide is SEQ ID NO: 8 or SEQ ID NO: 9.

15. The method of claim 5, wherein the inhibitor is either encapsulated in or operatively attached to a lipid particle, a liposome, a vesicle, a nanosphere, or a nanoparticle or the like and formulated for delivery to subject in vivo.

16. A method of preventing malignant transformation of a cell, comprising: providing a SATB1 inhibitor, and delivering a therapeutically effective amount of said compound to said cell.

17. (canceled)

18. The method of claim 1, wherein the cancer is small lung cell carcinoma, leukemia, lymphoma, bone or colon cancer.

19.-24. (canceled)


SEQUENCE LISTING (10 sequences)

SEQ ID NO: 1 - DNA, 295 bp, Homo sapiens
  unique sequence of special AT-rich sequence binding protein 1 (SATB1)
gtggaagccttgggaatcctccagagtttcatacaagacgtgggcctgtaccctgacgaagaggccatccagactctgtctgcccagctcgaccttcccaagtacaccatcatcaagttctttcagaaccagcggtactatctcaagcaccacggcaaactgaaggacaattccggtttagaggtcgatgtggcagaatataaagaagaggagctgctgaaggatttggaagagagtgtccaagataaaaatactaacacccttttttcagtgaaactagaagaagagctgtcag

SEQ ID NO: 2 - protein, 98 aa, Artificial Sequence
  synthetic SATB1 peptide fragment
VEALGILQSFIQDVGLYPDEEAIQTLSAQLDLPKYTIIKFFQNQRYYLKHHGKLKDNSGLEVDVAEYKEEELLKDLEESVQDKNTNTLFSVKLEEELS

SEQ ID NO: 3 - protein, 118 aa, Artificial Sequence
  synthetic SATB1 peptide fragment
SMIRRFLSLPQPERDAIYEQESNAVHHHGDRPPHIIHVPAEQIQQQQQQQQQQQQQQQAPPPPQPQQQPQTGPRLPPRQPTVASPAESDEENRQKTRPRTKISVEALGILQSFIQDVG

SEQ ID NO: 4 - DNA, 64 bp, Artificial Sequence
  synthetic SATB1 short hairpin RNA 1 (shRNA 1) sense sequence
gatccccggatttggaagagagtgtcttcaagagagacactctcttccaaatcctttttggaaa

SEQ ID NO: 5 - DNA, 64 bp, Artificial Sequence
  synthetic SATB1 short hairpin RNA 1 (shRNA 1) anti-sense sequence
agcttttccaaaaaggatttggaagagagtgtctctcttgaagacactctcttccaaatccggg

SEQ ID NO: 6 - DNA, 64 bp, Artificial Sequence
  synthetic SATB1 short hairpin RNA 2 (shRNA 2) sense sequence
gatccccgtccaccttgtcttctctcttcaagagagagagaagacaaggtggactttttggaaa

SEQ ID NO: 7 - DNA, 64 bp, Artificial Sequence
  synthetic SATB1 short hairpin RNA 2 (shRNA 2) anti-sense sequence
agcttttccaaaaagtccaccttgtcttctctctctcttgaagagagaagacaaggtggacggg

SEQ ID NO: 8 - DNA, 19 bp, Artificial Sequence
  synthetic SATB1 short hairpin RNA-2423 (shRNA-2423) oligoduplex target
ggatttggaagagagtgtc

SEQ ID NO: 9 - DNA, 19 bp, Artificial Sequence
  synthetic SATB1 short hairpin RNA-2595 (shRNA-2595) oligoduplex target
gtccaccttgtcttctctc

SEQ ID NO: 10 - protein, 6 aa, Artificial Sequence
  Factor Xa cleavage site
MHIEGR